(19) United States
(12) Patent Application Publication
(10) Pub. No.: US 2024/0158799 A1
(43) Pub. Date: May 16, 2024

(54) APTAMERS THAT SELECTIVELY BIND TO A SARS-COV-2 VIRUS NUCLEOCAPSID PROTEIN

(71) Applicants: Saimon Moraes Silva, Sandringham, Victoria (AU); IQ SCIENCE LIMITED, Lower Hutt (NZ)

(72) Inventors: Shalen Kumar, Lower Hutt (NZ); Angel Brito, Wellington (NZ); Laura Trobiani, Wellington (NZ); Saimon Moraes Silva, Sandringham (AU)

(21) Appl. No.: 18/278,721

(22) PCT Filed: Feb. 24, 2022
(86) PCT No.: PCT/NZ2022/050020

§ 371 Date: Aug. 24, 2023

Related U.S. Application Data
(60) Provisional application No. 63/152,953, filed on Feb. 24, 2021.

(51) Int. Cl. C12N 15/115; G01N 33/543; G01N 33/569

(52) U.S. Cl. CPC C12N 15/115; G01N 33/5438; G01N 33/56983; C12N 2310/16; G01N 2333/165

(57) ABSTRACT

The present invention is concerned with the detection of a SARS-Cov-2 vims antigen including, for example, a SARS-Cov-2 virus nucleocapsid protein and/or a SARS-Cov-2 virus spike protein. The present invention provides novel polynucleotide sequences which spontaneously fold to form aptamers having secondary structure features that promote selective binding to a SARS-CoV-2 virus antigen. The present invention further provides test kits and assay methods which employ the polynucleotides described herein, to achieve a more accurate, lower cost, rapid diagnosis COVID-19 test intended to accelerate contact tracing and testing of individuals and the community in the management of the ongoing global pandemic caused by various strains of SARS-CoV-2 virus.

### TECHNICAL FIELD

The present invention is concerned with the detection of SARS-Cov-2 virus (also known as COVID-19) using molecular tools which target virus antigens or antibodies that are made by the immune system in response to the presence of the virus. The present invention provides novel polynucleotide sequences which adopt a specific conformation to selectively bind the nucleocapsid protein (N) and spike protein (S) of SARS-Cov2 virus. Also provided herein are test kits and assay methods which employ the polynucleotide sequences for the detection of SARS-Cov-2 virus or virus fragments from a test sample including (e.g.) biological samples such as saliva or blood, or swabs obtained from (e.g.) healthcare environments such as hospitals and care homes, schools and domestic environments, public transport as well as sewage and waste water treatment plants.

### BACKGROUND OF THE INVENTION

Coronavirus disease 2019 (COVID-19) started as an epidemic in 2019, and since early 2020 has been regarded as a global pandemic. The virus responsible is called severe acute respiratory syndrome coronavirus 2 (SARS-CoV-2), a novel coronavirus sharing high sequence identity with bat- and pangolin-derived SARS-like coronaviruses, suggesting a zoonotic origin. SARS-CoV-2 is an enveloped, positive-sense, single-stranded RNA virus that can infect a broad range of vertebrates. The envelope has various proteins protruding outward such as the spike protein giving the overall look of a crown, hence the name “Corona”. The spike protein complex (composed of S1 and S2 subunits) are responsible for interacting with the human ACE2 receptors on the surface of the respiratory tract and causing acute respiratory distress syndrome (ARDS). The highest risk factors for mortality include advanced age, ethnicity, obesity, diabetes, and hypertension among other co-morbidities.

The current ‘gold standard’ diagnosis available for the detection of SARS-CoV-2 involves quantitative PCR (qPCR) testing which targets viral genes. However, there is a need to provide more rapid, lower-cost testing solutions which accelerate the rate at which mass-testing may generate definitive diagnoses in the management of the pandemic.

### SUMMARY OF THE INVENTION

The inventions described and claimed herein have many attributes and embodiments including, but not limited to, those set forth or described or referenced in this Summary of the Invention. It is not intended to be all-inclusive and the inventions described and claimed herein are not limited to or by the features or embodiments identified in this Summary of the Invention, which is included for purposes of illustration only and not restriction.

In an aspect of the present invention there is provided a polynucleotide or salt thereof which binds to a SARS-CoV-2 virus nucleocapsid protein, the polynucleotide or salt thereof consisting in a sequence having between about 30 and about 85 nucleotides and further comprising a sequence that has at least 75, 80, 85, 86, 87, 88, 89, 90, 91, 92, 93, 94, 95, 96, 97, 98, 99 or 100% sequence identity to any one of SEQ ID Nos: 2, 4, 6, 8, 10, 12 and 14.

In another aspect of the present invention there is provided a polynucleotide or salt thereof comprising the sequence TAACCACATAACCGCAAGA[Y]TATTGTGCTACTCTCCTCGT (SEQ ID NO: 61) where Y is any sequence comprising or consisting in a sequence that has at least 75, 80, 85, 86, 87, 88, 89, 90, 91, 92, 93, 94, 95, 96, 97, 98 or 99% sequence identity to any one of SEQ ID Nos: 2, 4, 6, 8, 10, 12 and 14, which polynucleotide or salt thereof selectively binds to a SARS-CoV-2 virus nucleocapsid protein.

In a further aspect of the present invention there is provided a polynucleotide or salt thereof comprising the sequence TAACCACATAACCGCAAGA[Y]TATTGTGCTACTCTCCTCGT (SEQ ID NO: 61), where Y is any sequence comprising or consisting in a sequence selected from any one of SEQ ID Nos: 2, 4, 6, 8, 10, 12 and 14, which polynucleotide or salt thereof selectively binds to a SARS-CoV-2 virus nucleocapsid protein.

In yet another aspect of the present invention there is provided a polynucleotide or salt thereof which binds to a SARS-CoV-2 virus spike protein, the polynucleotide or salt thereof consisting in a sequence having between about 30 and about 85 nucleotides and further comprising a sequence that has at least 75, 80, 85, 86, 87, 88, 89, 90, 91, 92, 93, 94, 95, 96, 97, 98, 99 or 100% sequence identity to any one of SEQ ID Nos: 16, 18, 20, 22, 24, 26, 28, 30, 32, 34, 36, 38, 40, 42, 44, 46, 48, 50, 52, 54, 56, 58 and 60.

In yet a further aspect of the present invention there is provided a polynucleotide or salt thereof comprising the sequence TAACCACATAACCGCAAGA[Y]TATTGTGCTACTCTCCTCGT

(SEQ ID NO: 61) where Y is any sequence comprising or consisting in a sequence that has at least 75, 80, 85, 86, 87, 88, 89, 90, 91, 92, 93, 94, 95, 96, 97, 98 or 99% sequence identity to any one of SEQ ID Nos: 16, 18, 20, 22, 24, 26, 28, 30, 32, 34, 36, 38, 40, 42, 44, 46, 48, 50, 52, 54, 56, 58 and 60, which polynucleotide or salt thereof selectively binds to a SARS-CoV-2 virus spike protein.

In a further aspect of the present invention there is provided a polynucleotide or salt thereof comprising the sequence TAACCACATAACCGCAAGA[Y]TATTGTGCTACTCTCCTCGT (SEQ ID NO: 61), where Y is any sequence comprising or consisting in a sequence selected from any one of SEQ ID Nos: 16, 18, 20, 22, 24, 26, 28, 30, 32, 34, 36, 38, 40, 42, 44, 46, 48, 50, 52, 54, 56, 58 and 60, which polynucleotide or salt thereof selectively binds to a SARS-CoV-2 virus spike protein.

In yet a further aspect of the present invention there is provided a test kit or article of manufacture for detecting the presence of a SARS-CoV-2 virus antioen in a test sample, the test kit or article of manufacture comprising a polynucleotide or salt thereof comprising or consisting in a sequence defined by any one of SEQ ID Nos: 1-60, and optionally instructions for how to detect the presence of a SARS-CoV-2 virus antigen in the test sample.

In yet a further aspect of the present invention there is provided a method for detecting the presence of a SARS-CoV-2 virus antigen in a test sample, the method comprising the steps of:
(i) contacting a test sample with a polynucleotide or salt thereof comprising or consisting in a sequence defined by any one of SEQ ID Nos: 1-60; and
(ii) measuring binding between the polynucleotide and a SARS-CoV-2 virus antigen from the test sample,
wherein, a measured binding interaction between the polynucleotide and a SARS-CoV-2 virus antigen reflects the presence of SARS-CoV-2 virus in the test sample.

### BRIEF DESCRIPTION OF THE FIGURES

FIG. 1 lists full-length aptamer sequences according to the present invention which selectively target and bind to the nucleocapsid protein of SARS-CoV-2 virus.

FIG. 2 lists ligand binding domain sequences derived from full-length aptamers which selectively target and bind to the nucleocapsid protein of SARS-CoV-2 virus,

FIG. 3 lists the polynucleotide/aptamer sequences according to the present invention which selectively target and bind to the spike protein of SARS-CoV-2 virus, including subunits S1 and S2.

FIG. 4 lists ligand binding domain sequences derived from full-length aptamers which selectively target and bind to the spike protein of SARS-CoV-2 virus, including subunits S1 and S2.

FIG. 5 shows a phylogenetic tree generated using Clustal Omega, depicting the structural relationship of the polynucleotide/aptamer sequences which selectively bind to SARS-CoV-2 virus nucleocapsid protein, as disclosed herein. Sequence distance scores are indicated against each sequence name.

FIG. 6 shows a phylogenetic tree generated using Clustal Omega, depicting the structural relationship of the polynucleotide/aptamer sequences which selectively bind to SARS-CoV-2 virus spike proteins, as disclosed herein. Sequence distance scores are indicated against each sequence name.

FIGS. 7A-7C show binding kinetic data for lead polynucleotide/aptamer sequences which selectively bind to SARS-CoV-2 virus nucleocapsid protein, as disclosed herein. FIG. 5A: polynucleotide/aptamer sequence designated “N06” with a Kd of 25.06 nM; FIG. 5B: polynucleotide/aptamer sequence designated “N08” with a Kd of 18.70 nM; FIG. 5C: polynucleotide/aptamer sequence designated “N11” with a Kd of 29.60 nM. FIG. 5D: polynucleotide/aptamer sequence designated “N09”, where binding saturation was not reached due to limitations on the concentration of target protein present in the test sample. However, these data clearly show N09 aptamer/polynucleotide binding to the nucleocapsid protein.

FIGS. 8A-8G show binding kinetic data for lead polynucleotide/aptamer sequences which selectively bind to SARS-CoV-2 virus spike protein, as disclosed herein. FIG. 6A: polynucleotide/aptamer sequence designated “5504”; FIG. 6B: polynucleotide/aptamer sequence designated “SS08”; FIG. 6C: polynucleotide/aptamer sequence designated “SS10”; FIG. 6D: polynucleotide/aptamer sequence designated “SS12”; FIG. 6E: polynucleotide/aptamer sequence designated “SS18”; FIG. 6F: polynucleotide/aptamer sequence designated “SS20”; FIG. 6G: polynucleotide/aptamer sequence designated “SS48”. Binding saturation for each of the polynucleotide/aptamer sequences was not reached due to limitations associated with the concentration of target protein present in the test sample. However, these data clearly show aptamer/polynucleotide binding to the S1/S2 spike complex.

FIG. 9 shows a sandwich Sybr green assay demonstrating specific displacement of Sybr Green dye using N11 capture aptamer (SEQ ID NO: 13) and N08 detection aptamer (SEQ ID NO: 7) in the presence of 1 ng/mL and 50 ng/mL SARS-Cov-2 nucleocapsid protein.

FIG. 10 shows a single aptamer Sybr green assay demonstrating specific displacement of Sybr Green dye using the N08 capture aptamer in the presence of 1 ng/mL and 50 ng/mL SARS-Cov-2 nucleocapsid protein.

FIG. 11 shows a sandwich ELONA assay using N11 capture aptamer (SEQ ID NO: 13) and N08 detection aptamer (SEQ ID NO: 7) in the presence of various concentrations of SARS-Cov-2 nucleocapsid protein, as described in Example 4. These data clearly reflect the specific binding interaction by these aptamer/polynucleotide sequences for nucleocapsid protein in a dose dependent manner.

FIG. 12 shows a lateral flow assay involving gold nanoparticle-aptamer complexes in the presence of 5′biotin modified N08 detection aptamer (SEQ ID NO: 7) complexed with avidin protein, as described in Example 5. The capture aptamer used in these experiments was N11 (SEQ ID NO: 13). The test and control strip lines are indicated.

FIG. 13 shows the extent of aptamer structure switching that occurs when the N06, N08 and N11 aptamers of the present invention bind a SARS-Cov-2 nucleocapsid protein antigen, as evidenced by the circular dichroism plot.

FIGS. 14 and 14B show logarithmic and linear plots, respectively, of nucleocapsid protein detection using the N06 (SEQ ID NO: 3) and N08 (SEQ ID NO: 7) aptamers according to the methodology outlined in Example 6.

FIG. 15 is a schematic of a sensing mechanism using the N06 aptamer (SEQ ID NO: 3) for detection of SARS-Cov-2 nucleocapsid protein using the Universal Biosensor Pty Ltd assay platform.

FIG. 16 shows a cyclic voltammogram recorded in saliva at 200 mVs⁻¹ for the N06 aptamer (SEQ ID NO: 3) immobilized on a proprietary test strip developed by Universal Biosensors Pty Limited. Chemistry deposition conditions: the N06 aptamer was deposited on the gold strip in the concentration of 1 μM, followed by deposition of 0.1% v/v 6-Mercapto-1-hexanol (MCH). The strip configuration comprises of gold as working electrode, gold as counter electrode and palladium as the reference electrode.

FIG. 17(a) shows a cyclic voltammogram recorded in saliva for the N06 aptamer (SEQ ID NO: 3) immobilized on the Universal Biosensors' test strip using the following scan rates: 100, 150, 200, 250, 300, 350, 400, 450, and 500 mVs⁻¹. Chemistry deposition conditions: the N06 aptamer was deposited on the Au strip in the concentration of 1 μM, followed by deposition of 0.1% v/v 6-Mercapto-1-hexanol (MCH). The strip configuration comprises of gold as working electrode, gold as counter electrode and palladium as reference electrode. FIG. 17(b) plots different scan rates versus anodic and cathodic peak currents for the N06 aptamer immobilized on the gold strip.

FIG. 18 shows a square wave voltammogram recorded in saliva at 100 Hz for the N06 aptamer (SEQ ID NO: 3) immobilized on the Universal Biosensors test strip. In this measurement is presented the result for five different strips prepared in the same conditions. Chemistry deposition conditions: the N06 aptamer was deposited on the gold strip in the concentration of 1 μM, followed by deposition of 0.1% v/v 6-Mercapto-1-hexanol (MCH). The strip configuration comprises of gold as working electrode, gold as counter electrode and palladium as reference electrode.

FIG. 19(a) shows square-wave voltammograms for the N06 aptamer (SEQ ID NO: 3) electrode strips (frequency 100 Hz, pulse amplitude 25 mV) obtained before (black line) and after spiking different concentration of COVID-19 N protein (1.4E⁻¹⁶, 1.4E⁻¹⁵, 1.4E⁻¹⁴, 1.4E⁻¹³, and 1.4E⁻¹² g/mL) solution in saliva sample from a healthy donor. Background peak currents were subtracted from the square-wave voltammograms to give the presented results. As the theoretical quantity of redox reporters does not change by the binding to the target N protein, suppression in the current subsequent to binding suggests a mechanism by which recognition of the target N protein alters the electronic communication between redox reporter and electrode surface. FIG. 19(b) shows binding-induced change in the square wave current after exposure of the sensor to different concentrations of N protein, obtained by serial dilution of a stock solution of COVID-19 N protein spiked in saliva. Each point is the mean of square wave peak currents of three independent electrodes ±standard errors (σ/√n).

FIG. 20 shows an interrogation of the selectivity of the N06 aptamer (SEQ ID NO: 3) sensor against common proteins presented in saliva. All experiments were carried out using saliva from a single healthy donor and spiking the interferent species with all of them resulting in concentration of 1E⁻⁹ g/ml. Error bars present the standard deviation for measurements done five different electrodes. Electrochemical response was obtained using square-wave voltammetry applying a frequency of 100 Hz and pulse amplitude of 25 mV.

FIG. 21 shows investigation of the N06 aptamer (SEQ ID NO: 3) sensor electrochemical response using saliva samples from four different healthy donors before and after spiking the samples with COVID N protein in the concentration of 1E-9 g/ml. Error bars present the standard deviation for measurements done five different electrodes. Electrochemical response was obtained using square-wave voltammetry applying a frequency of 100 Hz and pulse amplitude of 25 mV.

FIG. 22 shows investigation of the shelf-life of N06 aptamer (SEQ ID NO: 3) sensor. For these experiments, after preparation, the strips in two different conditions, in a desiccator under vacuum at ambient temperature (black bars) and inside a fridge at approximately 4-5° C. (crosshatching bars). Electrochemical responses were obtained using square-wave voltammetry by applying a frequency of 100 Hz and pulse amplitude of 25 mV. These experiments were carried out in PBS solution. As observed in this figure the electrochemical sensor achieved a response even after 7 weeks of storage.

### Definitions

### General Definitions

Unless specifically defined otherwise, all technical and scientific terms used herein are to be understood as having the same meanings as is understood by one of ordinary skill in the relevant art to which this disclosure pertains. Examples of definitions of common terms in medicine, molecular biology and biochemistry a can be found in Dictionary of Microbiology and Molecular Biology, Singleton et al, (2d ed. (1994); The Encyclopedia of Molecular Biology, Kendrew et al. (eds.), Blackwell Science Ltd., (1994); Molecular Biology and Biotechnology: a Comprehensive Desk Reference, Robert A. Meyers (ed.), VCH Publishers, Inc., (1995); The Dictionary of Cell & Molecular Biology, 4th Edition. Lackie. J (Ed.), Academic Press Inc. (2007), and The Oxford Dictionary of Biochemistry and Molecular Biology, 2nd edition, Cammack et al. (Eds.), Oxford University Press Inc. (2006).

It is also believed that practice of various examples of, encompassed by, and falling within the scope of the present invention can be performed using standard molecular biology and biochemistry protocols and procedures as known in the art, and as described, for example in “Current Protocols in Nucleic Acid Chemistry, Wiley Online Library, Various; Molecular Cloning: A Laboratory Manual, Maniatis et al., Cold Spring Harbor Laboratory Press, (1982); Molecular Cloning: A Laboratory Manual (2 ed.), Sambrook et al., Cold Spring Harbor Laboratory Press, (1989); Guide to Molecular Cloning Techniques Vol. 152, S. L. Berger and A. R. Kimmerl (Eds.), Academic Press Inc., (1987); Protein Synthesis and Ribosome Structure: Translating the Genome. Nierhaus, K and Wilson D (eds.), Wiley-VCH Inc. (2004); Synthetic Peptides: A User's Guide (Advances in Molecular Biology) 2nd edition, Grant G. (Ed.), Oxford University Press (2002); Remington: The Science and Practice of Pharmacy 21st edition, Beringer, P (Ed.), Lippincott Williams & Wilkins, (2005), pp. 2393; and other commonly available reference materials relevant in the art to which this disclosure pertains, and which are all incorporated by reference herein in their entireties.

It is intended that reference to a range of numbers disclosed herein (e.g. 1 to 10) also incorporates reference to all related numbers within that range (e.g. 1, 1.1, 2, 3, 3.9, 4, 5, 6, 6.5, 7, 8, 9 and 10) and also any range of rational numbers within that range (for example 2 to 8, 1.5 to 5.5 and 3.1 to 4.7) and, therefore, all sub-ranges of all ranges expressly disclosed herein are expressly disclosed. These are only examples of what is specifically intended and all possible combinations of numerical values between the lowest value and the highest value enumerated are to be considered to be expressly stated in this application in a similar manner.

The term “and/or”, e.g., “X and/or Y” shall be understood to mean either “X and Y” or “X or Y” and shall be taken to provide explicit support for both meanings or for either meaning.

The term “a” or “an” refers to one or more than one of the entity specified; for example, “a receptor” or “a nucleic acid molecule” may refer to one or more receptor or nucleic acid molecule, or at least one receptor or nucleic acid molecule. As such, the terms “a” or “an”, “one or more” and “at least one” can be used interchangeably herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### Selected Definitions

For the purposes of the present invention, the following terms shall have the following meanings.

The term “about” when used in connection with a referenced numeric indication means the referenced numeric indication plus or minus up to 10% of that referenced numeric indication. For example, “about 100” means from 90 to 110 and “about six” means from 5.4 to 6.6.

The terms “aptamer”, “polynucleotide”, “oilgonucleotide”, “nucleic acid” or “nucleic acid molecule” are used interchangeably herein to refer to a non naturally occurring nucleic acid which has a specific binding affinity for a target antigen. In certain examples, the aptamers described herein undergo a conformational change (referred to in the art as “structure switching”) on binding its target ligand or antigen.

The term “fragment” as used herein may be used interchangeably with the term “functional fragment” and means the same thing.

The term “functional fragment” as used herein means any part of a polynucleotide of the inventions disclosed herein that retains its ability to selectively bind to the same drug target as the polynucleotide from which it is derived.

The term “test kit” as used herein refers to an article of manufacture comprising various components to perform the assays and methods according to the inventions described herein.

The term “polynucleotide” as used herein refers to a deoxyribose nucleic acid (DNA) sequence, a ribose nucleic acid sequence (RNA), messenger ribose nucleic acid (mRNA) and complementary DNA (cDNA), and is comprised of a continuous sequence of two or more nucleotides, also referred to as “a nucleic acid sequence”.

The term “ligand” refers generally to any molecule that binds to a receptor, and includes without limitation, a polypeptide, a protein, a vitamin, a carbohydrate, a glycoprotein, a therapeutic agent, a drug, a glycosaminoglycan, or any combination thereof.

As used herein, “ligand” may include an antigen or a protein associated with the SARS-CoV-2 virus, or an antibody which has been generated by the immune system in response to the presence of the SARS-CoV-2 virus, and includes without limitation a variable heavy chain region (VH) or variable light chain region (VL).

The term “sample” as used herein refers to any sample for which it is desired to test for the presence SARS-CoV-2 virus. This includes, without limitation, a biological sample (e.g. saliva, blood etc), or swabs obtained from (e.g.) healthcare environments such as hospitals and care homes, schools and domestic environments, public transport and sewage and waste water treatment plants.

The term “reference threshold” as used herein means the level of assay activity measured in the absence of a test sample. In certain examples according to the inventions described herein, the reference threshold is determined using ethanol in place of test sample.

The term “detection means” as used herein refers to any apparatus, equipment or configuration adapted to detect the binding interaction between an aptamer/polynucleotide and its target SARS-CoV-2 virus protein or antibody. Examples of detection means include, but are not limited to, optical methods, spectroscopy, visible spectroscopy, Raman spectroscopy, UV spectroscopy, surface plasmon resonance, electrochemical methods, impedance, resistance, capacitance, mechanical sensing by changes in mass, changes in mechanical resonance, electrophoresis, gel electrophoresis, gel retardation, imaging, fluorescence and fluorescence resonance energy transfer, polymerase chain reaction etc.

The term “sequence identity” as used herein refers to the percentage of nucleotides in a candidate sequence(s) that are identical with the nucleotides in the sequence under interrogation after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as EMBOSS Pairwise Needle, BLAST, BLAST-2, ALIGN, CLUSTALW or Megalign (DNASTAR) software. For example, % nucleic acid sequence identity values can be generated using sequence comparison computer programs found on the European Bioinformatics Institute website (http://www.ebi.ac.uk).

### DETAILED DESCRIPTION

The present invention provides polynucloetides, test kits and assay methods for the detection of SARS-CoV-2 virus antigens including, without limitation, the nucleocapsid protein (N) and the spike protein complex, comprised of S1 and S2 subunits.

### Polynucleotides/Aptamers

The polynucleotides of the present invention spontaneously fold to form aptamers having secondary structure features that promote selective binding to an antigen associated with SARS-CoV-2 virus.

In an aspect of the present invention there is provided a polynucleotide or salt thereof comprising or consisting in the sequence TAACCACATAACCGCAAGA[X]ₙTATTGTGCTACTCTCCTCGT, where X is selected from A, T, U, G or C and n is an integer of between about 10 and about 120, in particular between about and about 45, which polynucleotide of salt thereof binds to a SARS-CoV-2 virus antigen. In an example according to this aspect of the present invention, the SARS-CoV-2 virus antigen is selected from a nucleocapsid protein and a spike protein antigen.

The polynucleotides of the present invention may also spontaneously fold to form aptamers having secondary structure features that promote selective binding to a human immunoglobulin molecule produced as a result of a SARS-CoV-2 virus infection.

Accordingly, in another aspect of the present invention there is provided a polynucleotide or salt thereof comprising or consisting in the sequence TAACCACATAACCGCAAGA[X]ₙTATTGTGCTACTCTCCTCGT, where X is selected from A, T, U, G or C and n is an integer of between about 10 and about 120, in particular between about and about 45, which polynucleotide of salt thereof binds to a a human immunoglobulin molecule produced as a result of a SARS-CoV-2 virus infection. In an example according to this aspect of the present invention, the human immunoglobulin molecule includes an antibody or antibody fragment specific for a SARS-CoV-2 antigen. In a further example according to this aspect of the present invention, the antibody fragment is selected from a variable heavy chain (VH) or a variable light chain (VL) of the antibody.

### SARS-CoV-2 Nucleocapsid Protein Aptamers

In reference to the Examples and Figures which follow, Applicant has developed aptamer sequences which are highly selective for the nucleocapsid protein derived from SARS-CoV-2 virus, with minimal cross-reactivity to other non-virus antigens.

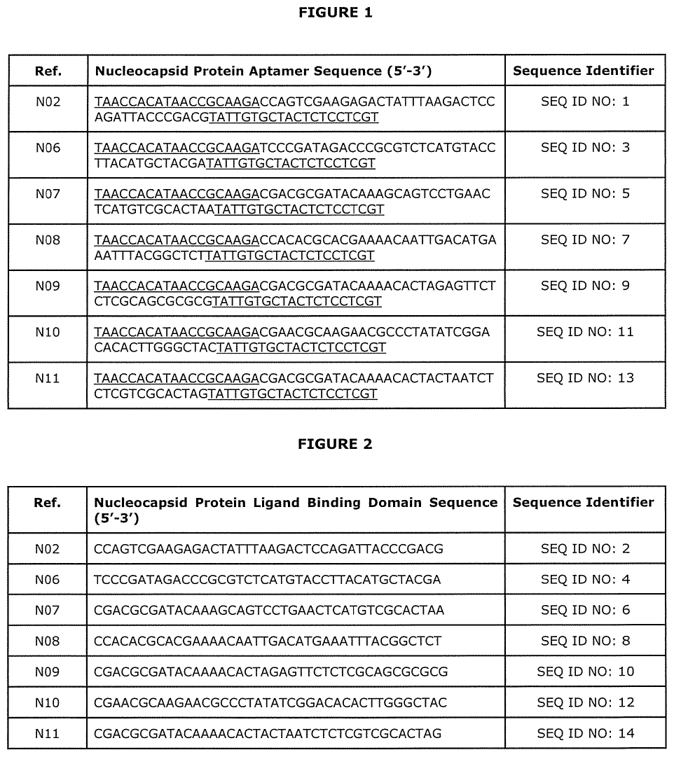

By way of illustration only, in reference to FIGS. 1, 5 and 7, Applicant developed aptamer sequences which selectively bind to a SARS-CoV-2 virus nucleocapsid protein.

Accordingly, in an aspect of the present invention there is provided a polynucleotide or salt thereof which binds to a SARS-CoV-2 virus nucleocapsid protein, the polynucleotide or salt thereof consisting in a sequence having between about 30 and about 85 nucleotides and further comprising a sequence that has at least 75, 80, 85, 86, 87, 88, 89, 90, 91, 92, 93, 94, 95, 96, 97, 98, 99 or 100% sequence identity to any one of SEQ ID Nos: 2, 4, 6, 8, 10, 12 and 14.

In yet another aspect of the present invention there is provided a polynucleotide or salt thereof comprising the sequence TAACCACATAACCGCAAGA[Y]TATTGTGCTACTCTCCTCGT (SEQ ID NO: 61) where Y is any sequence comprising or consisting in a sequence that has at least 75, 80, 85, 86, 87, 88, 89, 90, 91, 92, 93, 94, 95, 96, 97, 98 or 99% sequence identity to any one of SEQ ID Nos: 2, 4, 6, 8, 10, 12 and 14, which polynucleotide or salt thereof selectively binds to a SARS-CoV-2 virus nucleocapsid protein.

In another aspect of the present invention there is provided a polynucleotide or salt thereof comprising the sequence TAACCACATAACCGCAAGA[Y]TATTGTGCTACTCTCCTCGT (SEQ ID NO: 61), where Y is any sequence comprising or consisting in a sequence selected from any one of SEQ ID Nos: 2, 4, 6, 8, 10, 12 and 14, which polynucleotide or salt thereof selectively binds to a SARS-CoV-2 virus nucleocapsid protein.

In another aspect of the present invention there is provided a polynucleotide or salt thereof which selectively binds to a SARS-CoV-2 virus nucleocapsid protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 1 or a fragment thereof which retains selective binding for a SARS-Codi-2 virus nucleocapsid protein. In an example according to this aspect of the present invention, the fragment comprises or consists in a sequence defined by SEQ ID NO: 2.

In another aspect of the present invention there is provided a polynucleotide or salt thereof which selectively binds to a SANS-CoV-2 virus nucleocapsid protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 3 or a fragment thereof which retains selective binding for a SANS-CoV-2 virus nucleocapsid protein. In an example according to this aspect of the present invention, the fragment comprises or consists in a sequence defined by SEQ ID NO: 4.

In another aspect of the present invention there is provided a polynucleotide or salt thereof which selectively binds to a SANS-CoV-2 virus nucleocapsid protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 5 or a fragment thereof which retains selective binding for a SARS-Coif-2 virus nucleocapsid protein. In an example according to this aspect of the present invention, the fragment comprises or consists in a sequence defined by SEQ ID NO: 6.

In another aspect of the present invention there is provided a polynucleotide or salt thereof which selectively binds to a SAPS-CoV-2 virus nucleocapsid protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 7 or a fragment thereof which retains selective binding for a SAPS-CoV-2 virus nucleocapsid protein. In an example according to this aspect of the present invention, the fragment comprises or consists in a sequence defined by SEQ ID NO: 8.

In another aspect of the present invention there is provided a polynucleotide or salt thereof which selectively binds to a SANS-CoV-2 virus nucleocapsid protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 9 or a fragment thereof which retains selective binding for a SARS-CoV-2 virus nucleocapsid protein. In an example according to this aspect of the present invention, the fragment comprises or consists in a sequence defined by SEQ ID NO: 10.

In another aspect of the present invention there is provided a polynucleotide or salt thereof which selectively binds to a SAPS-CoV-2 virus nucleocapsid protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 11 or a fragment thereof which retains selective binding for a SARS-CoV-2 virus nucleocapsid protein. In an example according to this aspect of the present invention, the fragment comprises or consists in a sequence defined by SEQ ID NO: 12.

In another aspect of the present invention there is provided a polynucleotide or salt thereof which selectively binds to a SANS-CoV-2 virus nucleocapsid protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 13 or a fragment thereof which retains selective binding for a SANS-CoV-2 virus nucleocapsid protein. In an example according to this aspect of the present invention, the fragment comprises or consists in a sequence defined by SEQ ID NO: 14.

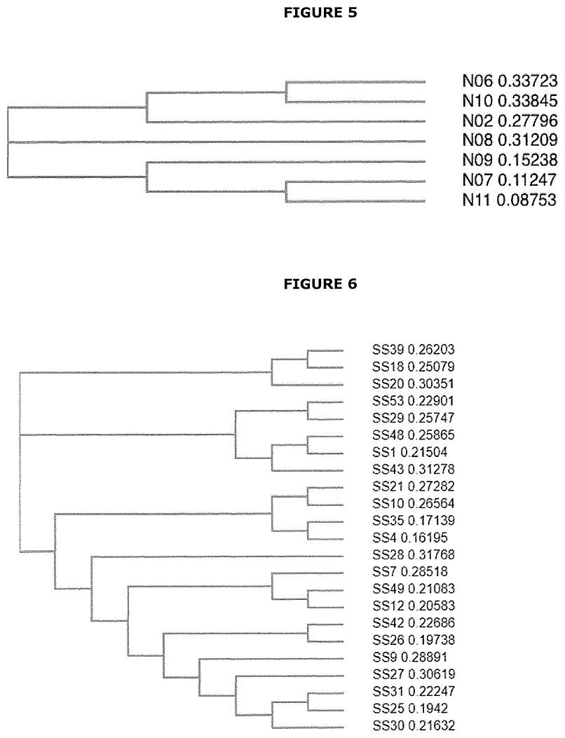

Cluster analysis was performed on the SARS-CoV-2 virus nucleocapsid protein aptamers disclosed herein using Clustal Omega Phylogenetic Trees. For further information refer to https//www.ebi.ac.uk/Tools/msa/clustalo/. The results from these analyses are presented in FIG. 5 where the level of structural (i.e. sequence) dis/similarity between the different aptamers is pictorially illustrated. By way of exemplification only, the SARS-CoV-2 virus nucleocapsid protein aptamers designated N06 and N10 share a higher degree of structural similarity to (e.g.) N02. However, N06, N10 and N02 collectively share a higher degree of structural similarity compared to (e.g.) N09, N07 and N11.

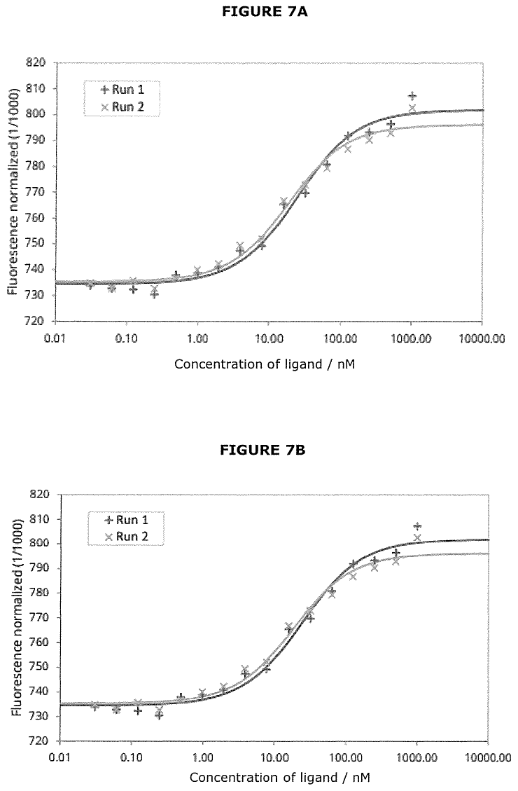

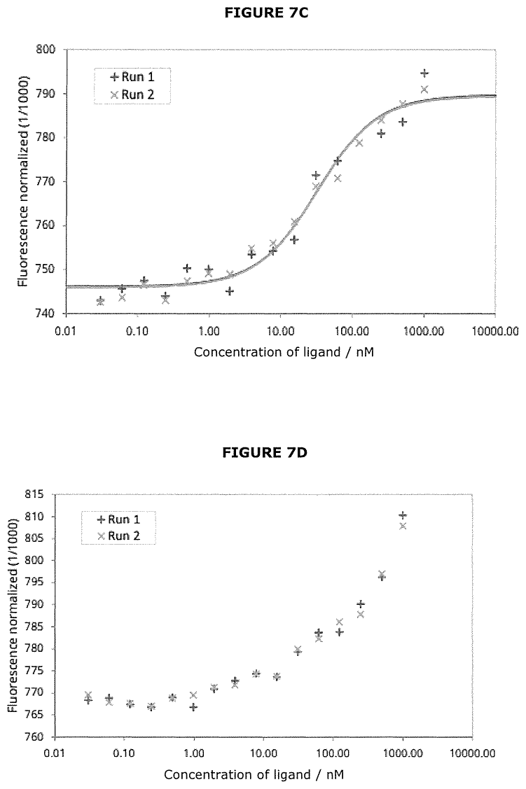

FIGS. 7A-7D provide dissociation constants for the aptamers designated N06 (SEQ ID NO: 3), N08 (SEQ ID NO: 7) and N11 (SEQ ID NO: 13) of Kds=25.5 nM, 18.7 nM and 29.6 nM, respectively, as measured using the methodologies described in the Examples. With dissociation constants in the medium nM range, these aptamers display highly selective binding affinity for a SARS-CoV-2 virus nucleocapsid protein.

Accordingly, the polynucleotides encoding SARS-CoV-2 virus nucleocapsid protein aptamers described herein, including without limitation SEQ ID NOs: 1, 3, 5, 7, 9, 11 and 13, display a binding affinity for a SARS-CoV-2 virus nucleocapsid protein which is between about 1 nM and about 1 μM, or about 1 nM and about 500 nM, or about 1 nM and about 200 nM.

By way of illustration only, the term “between about 1 nM and about 500 nM” includes a binding dissociation constant that is selected from 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 55, 50, 65, 60, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 405, 410, 415, 420, 425, 430, 435, 440, 445, 450, 452, 454, 456, 458, 460, 462, 464, 468, 470, 472, 474, 476, 478, 480, 482, 484, 486, 488, 490, 491, 492, 493, 494, 495, 496, 497, 498, 499 and 500 nM. The skilled person would, however, appreciate that “between about 1 nM and about 500 nM” extends beyond the definition presented above to include any whole integer within this range including, without limitation 53, 101, 207, 274, 318, 423, 479 nM etc.

### SARS-CoV-2 Spike Protein Aptamers

In reference to the Examples and Figures which follow, Applicant has developed aptamer sequences which are highly selective for spike protein/complex derived from SARS-CoV-2 virus, with minimal cross-reactivity to other non-virus antigens.

By way of illustration only, in reference to FIGS. 2, 6 and 8, Applicant developed aptamer sequences which selectively bind to a SARS-CoV-2 virus spike protein.

Accordingly, in an aspect of the present invention there is provided a polynucleotide or salt thereof which binds to a SARS-CoV-2 virus spike protein, the polynucleotide or salt thereof consisting in a sequence having between about 30 and about 85 nucleotides and further comprising a sequence that has at least 75, 80, 85, 86, 87, 88, 89, 90, 91, 92, 93, 94, 95, 96, 97, 98, 99 or 100% sequence identity to any one of SEQ ID Nos: 16, 18, 20, 22, 24, 26, 28, 30, 32, 34, 36, 38, 40, 42, 44, 46, 48, 50, 52, 54, 56, 58 and 60.

In yet another aspect of the present invention there is provided a polynucleotide or salt thereof comprising the sequence TAACCACATAACCGCAAGA[Y]TATTGTGCTACTCTCCTCGT (SEQ ID NO: 61) where Y is any sequence comprising or consisting in a sequence that has at least 75, 80, 85, 86, 87, 88, 89, 90, 91, 92, 93, 94, 95, 96, 97, 98 or 99% sequence identity to any one of SEQ ID Nos: 16, 18, 20, 22, 24, 26, 28, 30, 32, 34, 36, 38, 40, 42, 44, 46, 48, 50, 52, 54, 56, 58 and 60, which polynucleotide or salt thereof selectively binds to a SARS-CoV-2 virus spike protein.

In another aspect of the present invention there is provided a polynucleotide or salt thereof comprising the sequence TAACCACATAACCGCAAGA[Y]TATTGTGCTACTCTCCTCGT (SEQ ID NO: 61), where Y is any sequence comprising or consisting in a sequence selected from any one of SEQ ID Nos: 16, 18, 20, 22, 24, 26, 28, 30, 32, 34, 36, 38, 40, 42, 44, 46, 48, 50, 52, 54, 56, 58 and 60, which polynucleotide or salt thereof selectively binds to a SARS-CoV-2 virus spike protein.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 15 or a fragment thereof which retains selective binding for a SARS-CoV-2 virus spike protein. In an example according to this aspect of the present invention, the fragment comprises or consists in a sequence defined by SEQ ID NO: 16.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 17 or a fragment thereof which retains selective binding for a SARS-CoV-2 virus spike protein. In an example according to this aspect of the present invention, the fragment comprises or consists in a sequence defined by SEQ ID NO: 18.

In another aspect of the present invention there is provided a polynucleotide or salt thereof which selectively binds to a SARS-Codi-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 19 or a fragment thereof which retains selective binding for a SARS-CoV-2 virus spike protein. In an example according to this aspect of the present invention, the fragment comprises or consists in a sequence defined by SEQ ID NO: 20.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 21 or a fragment thereof which retains selective binding for a SARS-CoV-2 virus spike protein. In an example according to this aspect of the present invention, the fragment comprises or consists in a sequence defined by SEQ ID NO: 22.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 23 or a fragment thereof which retains selective binding for a SARS-CoV-2 virus spike protein. In an example according to this aspect of the present invention, the fragment comprises or consists in a sequence defined by SEQ ID NO: 24.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 25 or a fragment thereof which retains selective binding for a SARS-CoV-2 virus spike protein. In an example according to this aspect of the present invention, the fragment comprises or consists in a sequence defined by SEQ ID NO: 26.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 27 or a fragment thereof which retains selective binding for a SARS-CoV-2 virus spike protein. In an example according to this aspect of the present invention, the fragment comprises or consists in a sequence defined by SEQ ID NO: 28.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 29 or a fragment thereof which retains selective binding for a SANS-CoV-2 virus spike protein. In an example according to this aspect of the present invention, the fragment comprises or consists in a sequence defined by SEQ ID NO: 30.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 31 or a fragment thereof which retains selective binding for a SARS-CoV-2 virus spike protein. In an example according to this aspect of the present invention, the fragment comprises or consists in a sequence defined by SEQ ID NO: 32.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 33 or a fragment thereof which retains selective binding for a SARS-CoV-2 virus spike protein. In an example according to this aspect of the present invention, the fragment comprises or consists in a sequence defined by SEQ ID NO: 34.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 35 or a fragment thereof which retains selective binding for a SARS-CoV-2 virus spike protein. In an example according to this aspect of the present invention, the fragment comprises or consists in a sequence defined by SEQ ID NO: 36.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 37 or a fragment thereof which retains selective binding for a SARS-CoV-2 virus spike protein. In an example according to this aspect of the present invention, the fragment comprises or consists in a sequence defined by SEQ ID NO: 38.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 39 or a fragment thereof which retains selective binding for a SANS-CoV-2 virus spike protein. In an example according to this aspect of the present invention, the fragment comprises or consists in a sequence defined by SEQ ID NO: 40.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 41 or a fragment thereof which retains selective binding for a SARS-CoV-2 virus spike protein. In an example according to this aspect of the present invention, the fragment comprises or consists in a sequence defined by SEQ ID NO: 42.

In another aspect of the present invention there is provided a polynucleotide or salt thereof which selectively binds to a SARS-Codi-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 43 or a fragment thereof which retains selective binding for a SARS-CoV-2 virus spike protein. In an example according to this aspect of the present invention, the fragment comprises or consists in a sequence defined by SEQ ID NO: 44.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 45 or a fragment thereof which retains selective binding for a SARS-CoV-2 virus spike protein. In an example according to this aspect of the present invention, the fragment comprises or consists in a sequence defined by SEQ ID NO: 46.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 47 or a fragment thereof which retains selective binding for a SARS-CoV-2 virus spike protein. In an example according to this aspect of the present invention, the fragment comprises or consists in a sequence defined by SEQ ID NO: 48.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 49 or a fragment thereof which retains selective binding for a SARS-CoV-2 virus spike protein. In an example according to this aspect of the present invention, the fragment comprises or consists in a sequence defined by SEQ ID NO: 50.

In another aspect of the present invention there is provided a polynucleotide or salt thereof which selectively binds to a SARS-Codi-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 51 or a fragment thereof which retains selective binding for a SARS-CoV-2 virus spike protein. In an example according to this aspect of the present invention, the fragment comprises or consists in a sequence defined by SEQ ID NO: 52.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 53 or a fragment thereof which retains selective binding for a SARS-CoV-2 virus spike protein. In an example according to this aspect of the present invention, the fragment comprises or consists in a sequence defined by SEQ ID NO: 54.

In another aspect of the present invention there is provided a polynucleotide or salt thereof which selectively binds to a SARS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 55 or a fragment thereof which retains selective binding for a SARS-CoV-2 virus spike protein. In an example according to this aspect of the present invention, the fragment comprises or consists in a sequence defined by SEQ ID NO: 56.

In another aspect of the present invention there is provided a polynucleotide or salt thereof which selectively binds to a SAPS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 57 or a fragment thereof which retains selective binding for a SANS-CoV-2 virus spike protein. In an example according to this aspect of the present invention, the fragment comprises or consists in a sequence defined by SEQ ID NO: 58.

In another aspect of the present invention there is provided a polynucleotide or salt thereof which selectively binds to a SAPS-CoV-2 virus spike protein, the polynucleotide or salt thereof comprising or consisting in a sequence that has at least 75, 80, 85, 86, 87, 88, 89, 90, 91, 92, 93, 94, 95, 96, 97, 98, 99 or 100% sequence identity to SEQ ID NO: 59 or a fragment thereof which retains selective binding for a SAPS-CoV-2 virus spike protein. In an example according to this aspect of the present invention, the fragment comprises or consists in a sequence defined by SEQ ID NO: 60.

It will be appreciated by a person skilled in the art that the term “at least at least 75, 80, 85, 86, 87, 88, 89, 90, 91, 92, 93, 94, 95, 96, 97, 98, 99 or 100% sequence identity” or “at least at least 75, 80, 85, 86, 87, 88, 89, 90, 91, 92, 93, 94, 95, 96, 97, 98 or 99% sequence identity” also includes, without limitation, a sequence which has or shares at least 76, 77, 78, 79, 81, 82, 83 and 84% identity with the polynucleotide sequence recited.

Cluster analysis was performed on the SAPS-CoV-2 virus spike protein aptamers disclosed herein using Clustal Omega Phylogenetic Trees. For further information refer to https//www.ebi.ac.uk/Tools/msa/clustalo/. The results from these analyses are presented in FIG. 6 where the level of structural (i.e. sequence) dis/similarity between the different aptamers is pictorially illustrated. By way of exemplification only, the SAPS-CoV-2 virus spike protein aptamers designated SS39 and SS18 share a higher degree of structural similarity to (e.g.) SS53 and SS29. However, SS39, SS18, SS53 and SS29 collectively share a higher degree of structural similarity compared to (e.g.) SS31, SS25 and SS30.

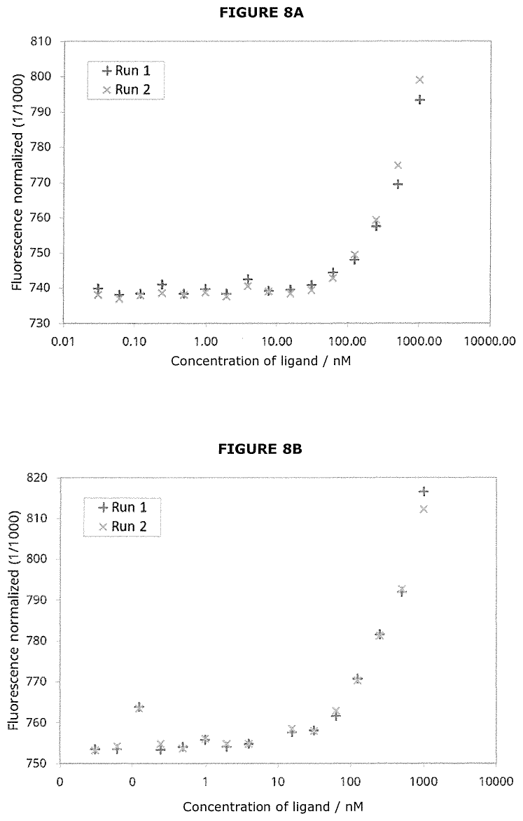

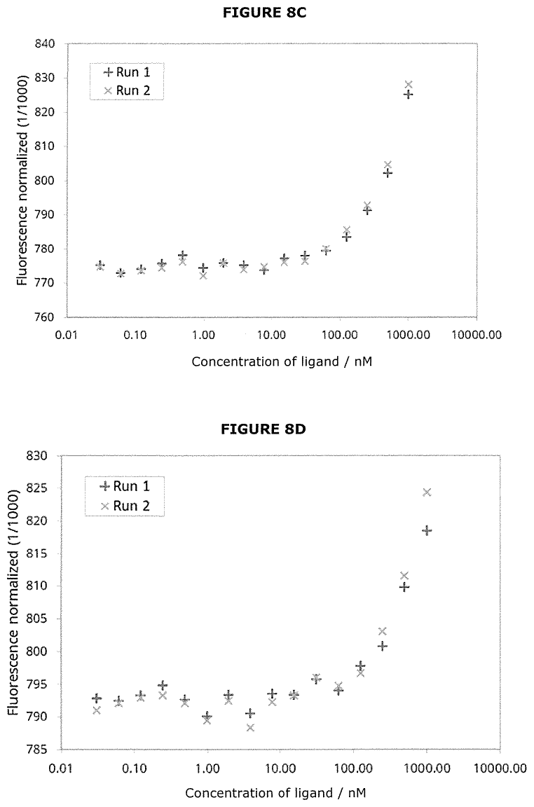

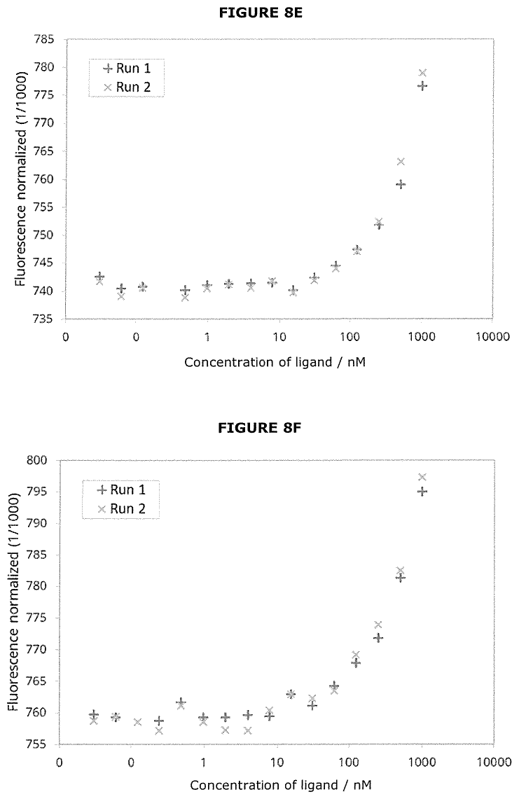

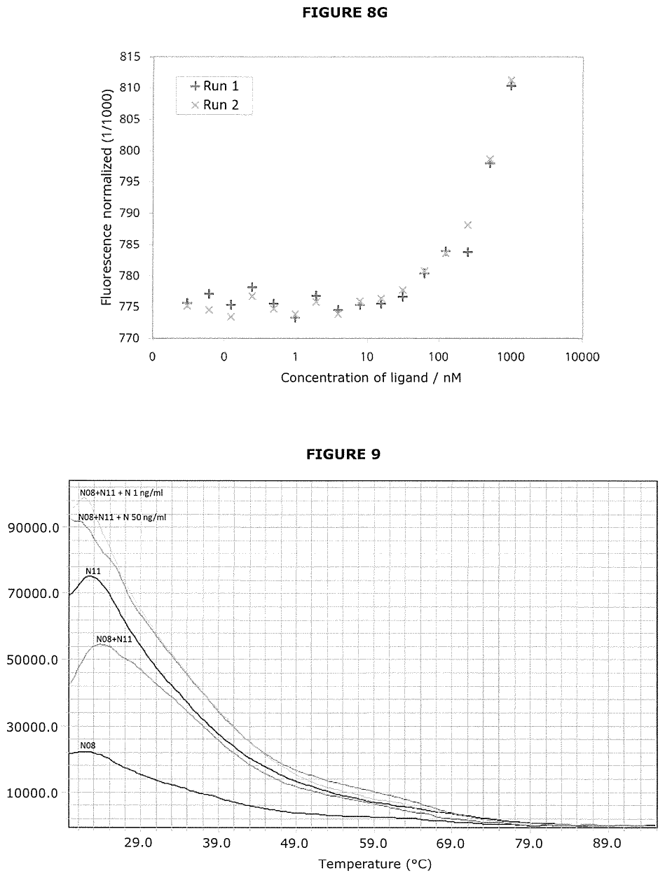

FIGS. 8A-8G provide preliminary kinetic binding data for aptamers designated SS04 (SEQ ID NO: 17), SS08 (SEQ ID NO: ?) and SS19 (SEQ ID NO: 23), SS12 (SEQ ID NO: 25), SS18 (SEQ ID NO: 27), SS20 (SEQ ID NO: 29) and SS48 (SEQ ID NO: 55). While binding saturation plateaus were not reached owing to limitations associated with the concentration of target protein present in the test sample. However, these data clearly show aptamer/polynucleotide binding to the S1/S2 spike complex.

Accordingly, the polynucleotides encoding SARS-CoV-2 virus spike protein aptamers described herein, including without limitation SEQ ID NOs: 15, 17, 19, 21, 23, 25, 27, 29, 31, 33, 35, 37, 39, 41, 43, 45, 47, 49, 51, 53, 55, 57 and 59, are anticipated to display a binding affinity for a SARS-CoV-2 virus spike protein which is between about 1 nM and about 1 μM, or about 1 nM and about 500 nM, or about 1 nM and about 200 nM.

Again, for any avoidance of doubt, and by way of illustration only the term “between about 1 nM and about 500 nM” includes a binding dissociation constant that is selected from 1, 2, 3, 4, 5, 6, 7, 8, 9, 10, 12, 14, 16, 18, 20, 22, 24, 26, 28, 30, 32, 34, 36, 38, 40, 42, 44, 46, 48, 50, 55, 50, 65, 60, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 405, 410, 415, 420, 425, 430, 435, 440, 445, 450, 452, 454, 456, 458, 460, 462, 464, 468, 470, 472, 474, 476, 478, 480, 482, 484, 486, 488, 490, 491, 492, 493, 494, 495, 496, 497, 498, 499 and 500 nM. The skilled person would, however, appreciate that “between about 1 nM and about 500 nM” extends beyond the definition presented above to include any whole integer within this range including, without limitation 53, 101, 207, 274, 318, 423, 479 nM etc.

### Methods for Detection of SARS-CoV92 Virus Antigens

The aptamer/polynucleotide sequences disclosed herein may be useful in performing assay methods for the detection of SARS-CoV-2 virus antigens. This may include, for example, contact trace testing to determine the infection state of an individual, border policing in managed quarantine facilities and/or to confirm that an individual presenting with symptoms of COVID-19 is indeed infected with a SARS-CoV-2 virus. Testing may also be used in hospitals etc to monitor progress using established or novel therapies.

According to the data presented in the various Examples and Figures disclosed herein, the nucleocapsid and spike protein aptamers according to the present invention may be incorporated in any detection system or assay platform familiar to a person skilled in the art. This includes, without limitation, a sandwich ELONA assay, a lateral flow assay, a SYBR green assay and, a nanoparticle assay or electrode/test strip assay, preferably involving gold. Refer to Examples 3-7 which follow.

Accordingly, in a further aspect of the present invention there is provided a method for determining the SARS-CoV-2 virus infection status of an individual or patient, the method comprising combining a test sample obtained from the individual with at least one labelled polynucleotide or salt thereof described, and using the results obtained from performance of the test kits to determine SARS-CoV-2 virus infection status of the individual or patient.

Accordingly, in a further aspect of the present invention there is provided a method for determining the SARS-CoV-2 virus infection status of an individual or patient, the method comprising combining a test sample obtained from the individual with a test kit or article of manufacture described herein, and using the results obtained from performance of the test kits to determine SARS-CoV-2 virus infection status of the individual or patient.

In certain examples according to the methods described herein, the method and test kit may configured to detect multiple SARS-CoV-2 virus antigens to reduce the number of false positive test outcomes. For example, detection of (e.g.) a nucleocapsid antigen and a spike protein antigen in the same test, or detection of (e.g.) a nucleocapsid antigen or a spike protein antigen in parallel with detection of a human immunoglobulin such as an antibody or antibody fragment.

According to the present invention, detection of at least one SARS-CoV-2 virus antigen is typically achieved by contacting a test sample with a polynucleotide (aptamer) selective for a SARS-CoV-2 virus antigen and measuring a binding interaction between the aptamer and the SARS-CoV-2 virus antigen, if present in the test sample.

Accordingly, in a further aspect of the present invention there is provided a method for detecting the presence of a SARS-CoV-2 virus antigen in a test sample, the method comprising the steps of:
(i) contacting a test sample with a polynucleotide or salt thereof comprising or consisting in a sequence defined by any one of SEQ ID Nos: 1-60; and
(ii) measuring binding between the polynucleotide and a SARS-CoV-2 virus antigen from the test sample,
wherein, a measured binding interaction between the polynucleotide and a SARS-CoV-2 virus antigen reflects the presence of SARS-CoV-2 virus in the test sample.

Alternatively, detection of at least one SARS-CoV-2 virus antigen is typically achieved by contacting a test sample with a polynucleotide (aptamer) selective for a human immunoglobulin produced as a result of a SARS-CoV-2 virus infection and measuring a binding interaction between the aptamer and the human immunoglobulin, if present in the test sample.

Accordingly, in a further aspect of the present invention there is provided a method for detecting the presence of a human immunoglobulin produced as a result of a SARS-CoV-2 virus infection in a test sample, the method comprising the steps of:
(i) contacting a test sample with a polynucleotide or salt thereof comprising or consisting a polynucleotide sequence defined herein; and
(ii) measuring binding between the polynucleotide and a human immunoglobulin produced as a result of a SARS-CoV-2 virus infection from the test sample,
wherein, a measured binding interaction between the polynucleotide and a human immunoglobulin produced as a result of a SARS-CoV-2 virus infection reflects the presence of SARS-CoV-2 virus in the test sample.

In certain examples according to the methods described herein, a binding interaction between the polynucleotide and the virus antigen or human immunoglobulin is measured using a detection means. Examples of detection means include, but are not limited, optical methods, spectroscopy, visible spectroscopy, Raman spectroscopy, UV spectroscopy, surface plasmon resonance, electrochemical methods, impedance, resistance, capacitance, mechanical sensing by changes in mass, changes in mechanical resonance, electrophoresis, gel electrophoresis, gel retardation, imaging, fluorescence, fluorescence resonance energy transfer.

### Lateral Flow and ELONA Assay Formats

The present invention also provides a test strip and/or lateral flow device comprising any aptamer or complex as described herein. Lateral flow devices may also be referred to as lateral flow tests, lateral flow assays and lateral flow immunoassays.

In certain examples, the lateral flow device comprises a support onto which an immobilisation oligonucleotide is attached. The immobilisation oligonucleotide is configured to hybridise to at least a portion of an immobilisation region of an aptamer as described herein. Any sample as described herein (e.g. a blood or plasma sample) may be introduced. If the sample comprises a SARS-Cov-2 antigen, the aptamer may bind to the SARS-Cov-2 antigen and undergo a conformational change, as evidenced by the circular dichroism data presented in FIG. 13.

In certain examples, the apparatus may be suitable for use in assays such as ELISA (enzyme-linked immunosorbent assay). When aptamers are used in place of antibodies, the resulting assay is often referred to as an “ELONA” (enzyme-linked oligonucleotide assay), “ELASA” (enzyme linked aptamer sorbent assay), “ELAA” (enzyme-linked aptamer assay) or similar. Incorporating aptamers into these ELISA-like assay platforms can result in increased sensitivity, allow a greater number of analytes to be detected; including analytes for which there are no antibodies available and a wide range of outputs, since aptamers can be conjugated to multiple reporter molecules including fluorophores, quencher molecules and/or any other detection moiety as described herein.

### Electrochemistry Assays

The present invention further contemplates electrochemistry assays in which the aptamers described herein are labelled with at least one redox active moiety and the aptamer is tethered to an electrode (e.g.) metal particle or metal plate/strip at the non-redox active group end. In its non-bound form (sensing interface not exposed to the target analyte), the proximity of the redox group to the electrode generates an electrochemical signal. When the aptamer binds its target N protein, the aptamer undergoes a conformational change (“structure switching”) and a change in electrochemical signal is obtained due to alteration in the electron transfer rate between redox active moiety and electrode surface. This change in electrochemical signal can be measured by using different voltammetric techniques (e.g.) square-wave voltammetry and cyclic voltammetry.

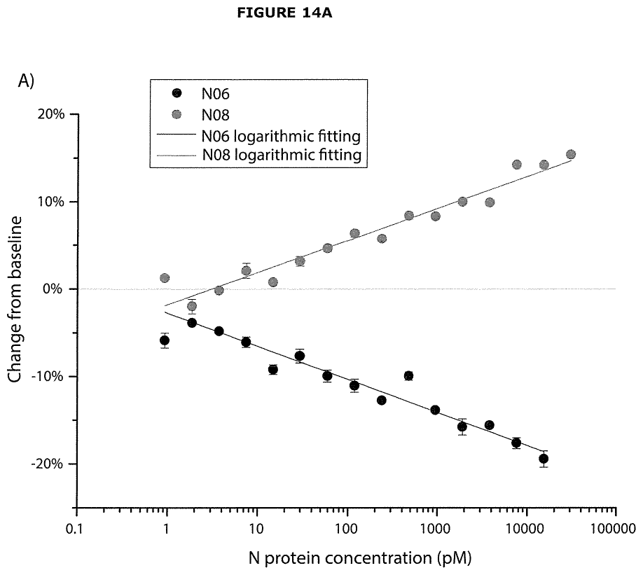

The data presented in Examples 6 and 7, read in conjunction with FIGS. 14-22, reflect certain embodiments of this general principle.

However, the skilled person would immediately recognize that any metal electrode when combined with an aptamer modified to include a redox active moiety as described herein could be used in an electrochemistry assay format for detection of SARS-Cov-2 biomarkers without the need for further experiment of invention.

### Test Kits & Articles of Manufacture

Further contemplated by the present invention is a test kit or article of manufacture suitable for performing a method or assay described herein, in particular for the detection of a SARS-Cov-2 antigen such as an antigen derived from the nucleocapsid protein or the spike protein.

In yet a further aspect of the present invention there is provided a test kit or article of manufacture for detecting the presence of a SARS-CoV-2 virus antigen in a test sample, the test kit or article of manufacture comprising a polynucleotide or salt thereof comprising or consisting in a sequence defined by any one of SEQ ID Nos: 1-60, and optionally instructions for how to detect the presence of a SARS-CoV-2 virus antigen in the test sample.

In certain examples according to the present invention, the test kits or articles of manufacture optionally include instructions for how to perform the detection of an illicit drug from the test sample interrogated.

### Exemplary Samples for Performance of Test Kits/Methods

The test kits and methods described herein may be performed on any test sample. In an example according to all aspects of the test kits, assays and methods described herein, the test sample is derived from biological material selected from the group consisting of urine, saliva, stool, hair, tissues including, but not limited to, blood (plasma and serum), muscle, tumors, semen, etc.

In yet a further example according to all aspects of the test kits, assays and methods described herein, the biological sample is derived from a human or an animal.

In yet a further example according to all aspects of the test kits, assays and methods described herein, the test sample includes a swab or a swipe obtained from a surface, including not limited to surfaces found within healthcare environments such as hospitals, doctor's surgeries, care homes, day clinics etc, surfaces found within schools, universities or sports grounds, surfaces in domestic environments such as semi/detached housing or common areas in flat complexes etc.

In yet a further example according to all aspects of the test kits, assays and methods described herein, the sample is derived from the environment including, without limitation, a liquid, water, plastics, sewage treatment plants etc.

In yet a further example according to all aspects of the test kits, assays and methods described herein, the test sample is derived from a food selected from the group consisting of vegetable, meat, beverage including but not limited to sports drink and milk, supplements including, but not limited to, food supplements and sports supplements, nutritional supplements, herbal extracts, etc.

### Modified Aptamers/Polynucleotides

Various modifications can be made to the polynucleotide aptamers disclosed herein to reduce exonuclease degradation, increase half-life for diagnostic applications, and/or for other purposes. Modification of the 3′end of the aptamer with inverted thymidine, deoxythymidine nucleotide, and polyethylene glycol (PEG) can reduce degradation of the oligonucleotide aptamer and increases stability of the aptamer. In some examples, PEG has an average molecular weight from about 20 to 80 kDa.

Further, the phosphodiester linkages of the deoxyribose-phosphate backbone of the aptamer can also be modified to improve stability.

In some examples, the aptamer is a polynucleotide comprising repeating units of the structure shown in Formula 1. Wavy lines demarcate one nucleotide and/or repeat unit from a neighboring nucleotide and/or repeat unit.

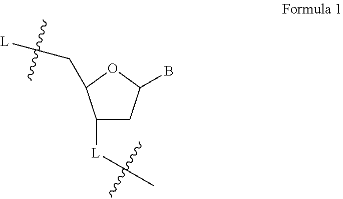

In some examples, each repeat unit of Formula 1 has a deoxyribose moiety linked to one of the common nucleotide bases (B), such as, adenosine, cytidine, guanosine, thymidine, and uridine. The base (B) for each repeating unit is independent from the other repeat units. The nucleotide sequences disclosed herein describe the order of appearance of bases (B) in an aptamer from the repeat unit on the 5′end of the aptamer to the 3′end of the aptamer.

In some examples, “L” is a linker group that links the deoxyribose moiety of adjacent repeat units. In the well-known structure of DNA, the L group is a phosphate group POOH, which can exist as a salt or in a neutral protonated form. The deoxyribose moiety together with the linker group forms the backbone of the aptamer, where the nucleotide base “B” varies independently of bonds and linkers between repeat units. Typically, the majority of the linker groups (L) forming the repeat units of Formula 1 in the aptamer are phosphate groups. As such, a majority of the backbone of the aptamer can be referred to as a deoxyribose-phosphate backbone. Many nuclease enzymes exist that can degrade oligonucleotide molecules without specificity for the specific nucleotide base sequence of the oligonucleotide molecule. Without wishing to be bound by any one particular theory, linker groups “L” other than phosphate can be incorporated into an oligonucleotide or aptamer to reduce or prevent degradation by nucleases.

In some examples, L can be replaced with a group as shown in Formula 2, where X₁₋₄ are independently O or S. X₂ and X₃ can be bonded to either the 3′carbon or the 5′carbon of a deoxyribose moiety. In some examples, X₁ is O and X₄ is O that can be either protonated or unprotonated. In some examples, one or more of X₂ and/or X₃ is S and X₁ and X₄ are O, where O can be either protonated or unprotonated. Where one of X₂ and/or X₃ are S, the aptamer can be referred to as having a thioester linkage in the deoxyribose-phosphate backbone.

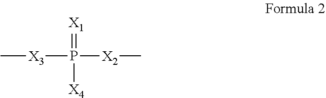

In some examples, the linker group “L” is an amide-containing group as shown in Formula 3, where R can be selected from hydrogen substituted or unsubstituted C₁-C₁₀ hydrocarbyl group. A “hydrocarbon” or “hydrocarbyl” refers to organic compounds or radicals consisting exclusively of the elements carbon and hydrogen. Hydrocarbyl includes alkyl, alkenyl, alkynyl, and aryl moieties. Hydrocarbyl also includes alkyl, alkenyl, alkynyl, and aryl moieties substituted with other aliphatic, cyclic or aryl hydrocarbon groups, such as alkaryl, alkenaryl and alkynaryl. In some examples, the linker group “L” is a group having Formula 3, and the aptamer comprises amide linkage (s) in the deoxyribose-phosphate backbone. The “NR” group of Formula 3 can be bonded to either the 3′carbon or the 5′carbon of a deoxyribose moiety. In some examples, R is methoxymethyl or methoxyethyl.

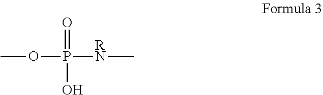

In some examples, the aptamer has from about 14 to about 100 nucleotide bases and/or repeat units, or any number of nucleotide bases or repeating units between 14 to 100, such as between 20 to 50 nucleotide bases and/or repeating units. In some examples, the aptamer has from about 14 to about 50 nucleotide bases and/or repeat units. In some examples, the aptamer has from about 30 to about 35 nucleotide bases and/or repeat units. In some examples, the aptamer has from 14 to 100, 14 to 95, 14 to 90, 14 to 85, 14 to 80, 14 to 75, 14 to 70, 14 to 65, 14 to 60, 14 to 55, 14 to 50, 14 to 45, 14 to 40, 14 to 35, 14 to 30, 14 to 25, 14 to 20, 14 to 18, 14 to 16, 14 to 15 nucleotide bases and/or repeating units, inclusive.

In some examples, the aptamer has from 20 to 100, 20 to 95, 20 to 90, 20 to 85, 20 to 80, 20 to 75, 20 to 70, 20 to 65, 20 to 60, 20 to 55, 20 to 50, 20 to 45, 20 to 40, 20 to 35, 20 to 30, 20 to 25 nucleotide bases and/or repeating units, inclusive. In some examples, the aptamer has from 25 to 100, 25 to 95, 25 to 90, 25 to 85, 25 to 80, 25 to 75, 25 to 70, to 65, 25 to 60, 25 to 55, 25 to 50, 25 to 45, 25 to 40, 25 to 35, 25 to 30 nucleotide bases and/or repeating units, inclusive. In some examples, the aptamer has from 30 to 100, 30 to 95, 30 to 90, 30 to 85, 30 to 80, 30 to 75, 30 to 70, 30 to 65, 30 to 60, 30 to 55, 30 to 50, to 45, 30 to 40, 30 to 35 nucleotide bases and/or repeating units, inclusive. In some examples, the aptamer has from 35 to 100, 35 to 95, 35 to 90, 35 to 85, 35 to 80, 35 to 75, to 70, 35 to 65, 35 to 60, 35 to 55, 35 to 50, 35 to 45, 35 to 40 nucleotide bases and/or repeating units, inclusive. In some examples, the aptamer has from 40 to 100, 40 to 95, 40 to 90, 40 to 85, 40 to 80, 40 to 75, 40 to 70, 40 to 65, 40 to 60, 40 to 55, 40 to 50, 40 to 45 nucleotide bases and/or repeating units, inclusive. In some examples, the aptamer has from to 100, 45 to 95, 45 to 90, 45 to 85, 45 to 80, 45 to 75, 45 to 70, 45 to 65, 45 to 60, 45 to 55, 45 to 50 nucleotide bases and/or repeating units, inclusive. In some examples, the aptamer has from 50 to 100, 50 to 95, 50 to 90, 50 to 85, 50 to 80, 50 to 75, 50 to 70, 50 to 65, 50 to 60, 50 to 55 nucleotide bases and/or repeating units, inclusive. In some examples, the aptamer has from 55 to 100, 55 to 95, 55 to 90, 55 to 85, 55 to 80, 55 to 75, 55 to 70, 55 to 65, 55 to 60 nucleotide bases and/or repeating units, inclusive. In some examples, the aptamer has from 60 to 100, 60 to 95, 60 to 90, 60 to 85, 60 to 80, 60 to 75, 60 to 70, 60 to 65 nucleotide bases and/or repeating units, inclusive. In some examples, the aptamer has 14, 15, 16, 18, 20, 25, 30, 35, 40, 45, 50, 55, 60, 65, 70, 75, 80, 85, 90, 95, or 100 nucleotide bases and/or repeating units.

In some examples, the aptamer comprises one or more repeating units having the linker “L” selected independently from Formulae 2 and 3. In some examples of the aptamer, the number of repeating units having the linker “L” selected independently from Formulae 2 and 3 is between 1 to 15 (inclusive), or any number there between, such as, for example, 1 to 10, 2 to 8, and 3 to 5, inclusive. In some examples of the aptamer, the number of repeating units having the linker “L” selected independently from Formulae 2 and 3 is between 1 to 15, 1 to 14, 1 to 12, 1 to 10, 1 to 9, 1 to 8, 1 to 7, 1 to 6, 1 to 5, 1 to 4, 1 to 3, 1 to 2, inclusive. In some examples of the aptamer, the number of repeating units having the linker “L” selected independently from Formulae 2 and 3 is 1, 2, 3, 4, 5, 6, 7, 8, 9, 10, 12, 14, or 15. Linker groups in repeat units not selected from Formulae 2 or 3 are phosphate.

In some examples of the aptamer, about 10% to about 100% (or any percentages there between, such as about 20% to about 90%, about 30% to about 50%) of the repeat units have the linker “L” selected independently from Formulae 2 and 3. In some examples of the aptamer, about 10% to about 70% of the repeat units have the linker “L” selected independently from Formulae 2 and 3. In some examples of the aptamer, about 10% to about 50% of the repeat units have the linker “L” selected independently from Formulae 2 and 3. In some examples of the aptamer, about 10% to about 30% of the repeat units have the linker “L” selected independently from Formulae 2 and 3. In some examples of the aptamer, about 10% to about 20% of the repeat units have the linker “L” selected independently from Formulae 2 and 3. Linker groups in repeat units not selected from Formulae 2 or 3 are phosphate.

Various nucleases are exonucleases that degrade oligonucleotides from the 5′ or 3′end. As such, in some examples, a linker group L selected from Formulae 2 or 3 is located within about 5 repeat units from the 5′ or the 3′end of the aptamer. In some examples, a linker group L selected from Formulae 2 or 3 is located within about 3 repeat units from the 5′ or the 3′end of the aptamer. In some examples, a linker group L selected from Formulae 2 or 3 is located within 3 repeat units from the 5′ or the 3′end of the aptamer. In some examples, a linker group L selected from Formulae 2 or 3 is located within 2 repeat units from the 5′ or the 3′ end of the aptamer. In some examples, a linker group L selected from Formulae 2 or 3 is part of the repeat unit on the 5′ or the 3′end of the aptamer.

Degradation of the aptamers can also be reduced by the inclusion of modified nucleotide bases (B). The pyrimidine nucleotide bases, cytosine, thymine and uracil can be replaced with alkylated pyrimidines. Examples of alkylated pyrimidines include pseudoisocytosine; N4, N4-ethanocytosine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; 1-methylpseudouracil; 3-methylcytosine; 5-methylcytosine; 5-methylaminomethyl uracil; 5-methoxy amino methyl-2-thiouracil; 5-methoxycarbonylmethyluracil; 5-methoxyuracil; uracil-5-oxyacetic acid methyl ester; pseudouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; 2-thiocytosine; 5-propyluracil; 5-propylcytosine; 5-ethyluracil; 5-ethylcytosine; 5-butyluracil; 5-pentyluracil; 5-pentylcytosine; methylpseudouracil; and 1-methylcytosine. The purine nucleotide bases, adenine and guanine, can be replaced by alkylated purines. Examples alkylated purines include 8-hydroxy-N6-methyladenine; inosine; N6-isopentyl-adenine; 1-methyladenine; 1-methylguanine; 2, 2-dimethylguanine; 2-methyladenine; 2-methylguanine; N6-methyladenine; 7-methylguanine; 2-methylthio-N6-isopentenyladenine; and 1-methylguanine.

In some examples, at least one deoxyribose or ribose of the nucleic acid aptamer is replaced with a morpholine ring. In one form, at least one phosphorothioate or phosphodiester linkage of the nucleic acid aptamer is replaced with phosphorodiamidate.

The nucleotides that can be substituted for natural nucleotides of DNA have a base moiety such as inosine, 5-fluorouracil, 5-bromouracil, hypoxanthine, 1-methylguanine, 5-methylcytosine, or tritylated bases. The sugar moiety of the nucleotide in a sequence can also be modified with a group such as arabinose, xylulose, or hexose. In addition, the adenine, cytosine, guanine, thymine, and uracil bases of the nucleotides can be modified with acetyl, methyl, hydroxyl, and thiol groups. Sequences containing nucleotide substitutions, deletions, and/or insertions can be prepared and tested using standard techniques known in the art.

The disclosed polynucleotide aptamers may be modified to promote an association with a substrate (e.g. particle or solid surface). In certain examples, the disclosed polynucleotide aptamers may be coupled or conjugated to one or more chemical entities or moieties to aid functionalization (e.g.) in a detection assay involving a substrate including but not limited to a particle or solid surface.

### Detection Labels

In certain examples, the aptamers of the invention are used to detect and/or quantify the amount of a SARS-Cov-2 antigen in a sample. Typically, the aptamers comprise a detectable label. Any label capable of facilitating detection and/or quantification of the aptamers may be used herein.

In certain examples, the detectable label is a fluorescent moiety, e.g. a fluorescent/quencher compound. Fluorescent/quencher compounds are known in the art. See, for example, Mary Katherine Johansson, Methods in Molecular Biol. 335: Fluorescent Energy Transfer Nucleic Acid Probes: Designs and Protocols, 2006, Didenko, ed., Humana Press, Totowa, NJ, and Marras et al., 2002, Nucl. Acids Res. 30, el22 (incorporated by reference herein).

In certain examples, the detectable label is FAM. In certain examples, the FAM-label is situated at the first or second primer region of the aptamer. The person skilled in the art would understand that the label could be located at any suitable position within the aptamer. Moieties that result in an increase in detectable signal when in proximity of each other may also be used herein, for example, as a result of fluorescence resonance energy transfer (“FRET”); suitable pairs include but are not limited to fluoroscein and tetramethylrhodamine; rhodamine 6G and malachite green, and FITC and thiosemicarbazole, to name a few.

In certain examples, the detectable label is selected from a fluorophore, a nanoparticle, a quantum dot, an enzyme, a radioactive isotope, a pre-defined sequence portion, a biotin, a desthiobiotin, a thiol group, an amine group, an azide, an aminoallyl group, a digoxigenin, an antibody, a catalyst, a colloidal metallic particle, a colloidal non-metallic particle, an organic polymer, a latex particle, a nanofiber, a nanotube, a dendrimer, a protein, and a liposome.

In certain examples, the detectable label is a fluorescent protein such as Green Fluorescent Protein (GFP) or any other fluorescent protein known to those skilled in the art.

In certain examples, the detectable label is an enzyme. For example, the enzyme may be selected from horseradish peroxidase, alkaline phosphatase, urease, b-galactosidase or any other enzyme known to those skilled in the art.

In certain examples, the nature of the detection will be dependent on the detectable label used. For example, the label may be detectable by virtue of its colour e.g. gold nanoparticles. A colour can be detected quantitatively by an optical reader or camera e.g. a camera with imaging software.

In certain examples, the detectable label is a fluorescent label e.g. a quantum dot. In such examples, the detection means may comprise a fluorescent plate reader, strip reader or similar, which is configured to record fluorescence intensity.

In examples in which the detectable label is an enzyme label, the detection means may, for example, be colorimetric, chemiluminescence and/or electrochemical (for example, using an electrochemical detector). Typically, electrochemical sensing is through conjugation of a redox reporter (e.g. methylene blue or ferrocene) to one end of the aptamer and a sensor surface to the other end. Typically, a change in aptamer conformation upon target binding changes the distance between the reporter and sensor to provide a readout.

In certain examples, the detectable label may further comprise enzymes such as horseradish peroxidase (HRP), alkaline phosphatase (APP) or similar, to catalytically turnover a substrate to give an amplified signal.

In certain examples, the invention provides a complex (e.g. conjugate) comprising aptamers of the invention and a detectable molecule. Typically, the aptamers of the invention are covalently or physically conjugated to a detectable molecule.

In certain examples, the detectable molecule is a visual, optical, photonic, electronic, acoustic, opto-acoustic, mass, electrochemical, electro-optical, spectrometric, enzymatic, or otherwise physically, chemically or biochemically detectable label.

In certain examples, the detectable molecule is detected by luminescence, UV/VIS spectroscopy, enzymatically, electrochemically or radioactively. Luminescence refers to the emission of light. For example, photoluminescence, chemiluminescence and bioluminescence are used for detection of the label. In photoluminescence or fluorescence, excitation occurs by absorption of photons. Exemplary fluorophores include, without limitation, bisbenzimidazole, fluorescein, acridine orange, Cy5, Cy3 or propidium iodide, which can be covalently coupled to aptamers, tetramethyl-6-carboxyhodamine (TAMRA), Texas Red (TR), rhodamine, Alexa Fluor dyes (et al. Fluorescent dyes of different wavelengths from different companies).

In certain examples, the detectable molecule is a colloidal metallic particle, e.g. gold nanoparticle, colloidal non-metallic particle, quantum dot, organic polymer, latex particle, nanofiber (e.g. carbon nanofiber), nanotube (e.g. carbon nanotube), dendrimer, protein or liposome with signal-generating substances. Colloidal particles can be detected colorimetrically.

In certain examples, the detectable molecule is an enzyme. In certain examples, the enzyme may convert substrates to coloured products, e.g. peroxidase, luciferase, b-galactosidase or alkaline phosphatase. For example, the colourless substrate X-gal is converted by the activity of b-galactosidase to a blue product whose colour is visually detected.

In certain examples, the detection molecule is a radioactive isotope. The detection can also be carried out by means of radioactive isotopes with which the aptamer is labelled, including but not limited to 3H, 14C, 32P, 33P, 35S or 125I, more preferably 32P, 33P or 125I. In the scintillation counting, the radioactive radiation emitted by the radioactively labelled aptamer target complex is measured indirectly. A scintillator substance is excited by the isotope's radioactive emissions. During the transition of the scintillation material, back to the ground state, the excitation energy is released again as flashes of light, which are amplified and counted by a photomultiplier.

In certain examples, the detectable molecule is selected from digoxigenin and biotin. Thus, the aptamers may also be labelled with digoxigenin or biotin, which are bound for example by antibodies or streptavidin, which may in turn carry a label, such as an enzyme conjugate. The prior covalent linkage (conjugation) of an aptamer with an enzyme can be accomplished in several known ways. Detection of aptamer binding may also be achieved through labelling of the aptamer with a radioisotope in an RIA (radioactive immunoassay), preferably with 125I, or by fluorescence in a FIA (fluoroimmunoassay) with fluorophores, preferably with fluorescein or FITC.

### Substrate/Solid Supports

In certain examples, the aptamer is attached to a support. Typically, the support is a solid support such as a membrane or a bead. The support may be a two-dimensional support e.g. a microplate or a three-dimensional support e.g. a bead. In certain embodiments, the support may comprise at least one magnetic bead.

In certain examples, the support may comprise at least one nanoparticle e.g. gold nanoparticles or the like. In yet further examples, the support comprises a microtiter or other assay plate, a strip, a membrane, a film, a gel, a chip, a microparticle, a nanofiber, a nanotube, a micelle, a micropore, a nanopore or a biosensor surface. In certain examples, the biosensor surface may be a probe tip surface, a biosensor flow-channel or similar.

In certain examples, the aptamer may be attached, directly or indirectly, to a magnetic bead, which may be e.g. carboxy-terminated, avidin-modified or epoxy-activated or otherwise modified with a compatible reactive group.

Immobilisation of aptamers to a support e.g. a solid phase support can be accomplished in a variety of ways and in any manner known to those skilled in the art for immobilising DNA or RNA on solids. For example, a solid phase of paper or a porous material may be wetted with the liquid phase aptamer, and the liquid phase subsequently volatilized leaving the aptamer in the paper or porous material.

In certain examples, the support comprises a membrane, e.g. a nitrocellulose, a polyethylene (PE), a polytetrafluoroethylene (PTFE), a polypropylene(PP), a cellulose acetate (CA), a polyacrylonitrile (PAN), a polyimide (PI), a polysulfone (PS), a polyethersulfone (PES) membrane or an inorganic membrane comprising aluminium oxide (Al₂O₃), silicon oxide (SIO₂) and/or zirconium oxide (ZrO). Particularly suitable materials from which a support can be made include for example inorganic polymers, organic polymers, glasses, organic and inorganic crystals, minerals, oxides, ceramics, metals, especially precious metals, carbon and semiconductors. A particularly suitable organic polymer is a polymer based on polystyrene. Biopolymers, such as cellulose, dextran, agar, agarose and Sephadex, which may be functionalized, in particular as nitrocellulose or cyanogen bromide Sephadex, can be used as polymers which provide a solid support.

### Aptamers—General

Aptamers are nucleic acid species that have been engineered through repeated rounds of in vitro selection equivalently, SELEX (systematic evolution of ligands by exponential enrichment) to bind to various molecular targets such as small molecules, proteins, nucleic acids, and even cells, tissues and organisms. Aptamers offer molecular binding and recognition equivalent to antibodies. In addition to their discriminate recognition, aptamers offer advantages over antibodies as they can be engineered completely in vitro, are readily produced by chemical synthesis, possess desirable storage properties, and elicit little or no immunogenicity in therapeutic applications.

According to an example of the present invention, the aptamer is a monomer (one unit). According to another example of the invention, the aptamer is a multimeric aptamer. The multimeric aptamer may comprise a plurality of aptamer units (mers). Each of the plurality of units of the aptamer may be identical. In such a case the multimeric aptamer is a homomultimer having a single specificity but enhanced avidity (multivalent aptamer).

Alternatively, the multimeric aptamer may comprise two or more aptameric monomers, wherein at least two mers of the multimeric aptamer are non-identical in structure, nucleic acid sequence or both. Such a multimeric aptamer is referred to herein as a heteromultimer. The heteromultimer may be directed to a single binding site i.e., monospecific (such as to avoid steric hindrance). The heteromultimer may be directed to a plurality of binding sites i.e., multispecific. The heteromultimer may be directed to a plurality of binding sites on different analytes, including for different SARS-CoV-2 virus antigens. Further description of the multimeric aptamer is provided below.

A plurality of multimeric aptamers may be conjugated to form a conjugate of multimeric aptamers. The multimeric aptamer may comprise, two (dimer), three (trimer), four (tetramer), five (pentamer), six (hexamer), and even more units.

Aptamers of the invention can be synthesized and screened by any suitable methods known in the art.

For example, aptamers can be screened and identified from a random aptamer library by SELEX (systematic evolution of ligands by exponential enrichment). Aptamers that bind to an antigen of interest can be suitably screened and selected by a modified selection method herein referred to as cell-SELEX or cellular-SELEX. In other examples, aptamers that bind to a cell surface target molecule can be screened by capillary electrophoresis and enriched by SELEX based on the observation that aptamer-target molecule complexes exhibited retarded migration rate in native polyacrylamide gel electrophoresis as compared to unbound aptamers.

A random aptamer library can be created that contains monomeric, dimeric, trimeric, tetrameric or other higher multimeric aptamers. A random aptamer library (either ssDNA or RNA) can be modified by including oligonucleotide linkers to link individual aptamer monomers to form multimeric aptamer fusion molecules. In other examples, a random oligonucleotide library is synthesized with randomized 45 nt sequences flanked by defined 20 nt sequences both upstream and downstream of the random sequence, i.e., known as 5′-arm and 3′-arm, which are used for the amplification of selected aptamers. A linking oligonucleotide (i.e., linker) is designed to contain sequences complementary to both 5′-arm and 3′-arm regions of random aptamers to form dimeric aptamers. For trimeric or tetrameric aptamers, a small trimeric or tetrameric (i.e., a Holiday junction-like) DNA nanostructure is engineered to include sequences complementary to the 3′-arm region of the random aptamers, therefore creating multimeric aptamer fusion through hybridization. In addition, 3-5 or 5-10 dT rich nucleotides can be engineered into the linker polynucleotides as a single stranded region between the aptamer-binding motifs, which offers flexibility and freedom of multiple aptamers to coordinate and synergize multivalent interactions with cellular ligands or receptors. Alternatively, multimeric aptamers can also be formed by mixing biotinylated aptamers with streptavidin.

A modified cellular SELEX procedure can be employed to select target-binding aptamers. Multimeric aptamers may be multivalent but be of single binding specificity (i.e., homomultimeric aptamers). Alternatively, the multimeric aptamer may be multivalent and multi-specific (i.e., heteromultimeric aptamers).

Thus, each monomer of the homomultimeric aptamer binds the target protein (e.g., selected drug target) in an identical manner. Thus according to an example of the invention, all monomeric components of the homomultimeric aptamer are identical.

Conversely, a heteromultimeric aptamer comprises a plurality of monomeric aptamers at least two of which bind different sites on a single target protein or bind at least two different target proteins.

Selection of DNA or RNA-aptamers is well-established using protocols described in the scientific literature.

In certain examples, a suitable nucleotide length for an aptamer ranges from about 15 to about 100 nucleotide (nt), and in various other examples, 12-30, 14-30, 15-30 nt, 30-100 nt, 30-60 nt, 25-70 nt, 25-60 nt, 40-60 nt, or 40-70 nt in length.

In other examples, the aptamer has affinity at the range of 10-100 nM, which, after binding of the aptamer to a molecule, permits dissociation of the aptamer from the target molecule, which leads to the release and recycle of the aptamer nucleic acid nanostructure. The affinity of individual aptamers can be increased by 4-50 fold by constructing multimeric aptamers linked together by covalent or non-covalent linkages. Methods of multimerizing aptamers are further described below.

Thus, in certain examples, the desirable affinity of an aptamer to an analyte of interest can be fine-tuned by adjusting the multiplexity of the monomeric aptamer.

Multimerization can be done at the library level as follows.

In certain examples, a linker polynucleotide has a length between about 5 nucleotides (nt) and about 100 nt; in various examples, 10-30 nt, 20-30 nt, 25-35 nt, 30-50 nt, 40-50 nt, 50-60 nt, 55-65 nt, 50-80 nt, or 80-100 nt. It is within the ability of one of skill in the art to adjust the length of the linker polynucleotide to accommodate each monomeric aptamer in the multimeric structure.

In certain examples, the multimeric aptamers can be identified and screened from a random multimeric aptamer library as described herein. In other examples, the monomeric aptamers are linked to each other by one or a plurality of linker polynucleotides to form multimeric aptamers. Monomeric aptamers can be linked to form multimeric aptamers by any suitable means and in any configurations.

It will be appreciated that the monomeric structures of the invention can be further multimerized by post SELEX procedures.

Multimers can be linearly linked by continuous linear synthesis of DNA without spacers or with nucleic acid spacers. Aptamer synthesis usually relies on standard solid phase phosphoramitide chemistry.

Thus, dimers, trimers and tetramers or higher oligomeric structures (e.g., pentamers, hexamers, heptamers, octamers etc.) can be linked by a polymeric spacer.

In certain examples, the aptamers are further modified to protect the aptamers from nuclease and other enzymatic activities. The aptamer sequence can be modified by any suitable methods known in the art. For example, phosphorothioate can be incorporated into the backbone, and 5′-modified pyrimidine can be included in 5′ end of ssDNA for DNA aptamer. For RNA aptamers, modified nucleotides such as substitutions of the 2′—OH groups of the ribose backbone, e.g., with 2′-deoxy-NTP or -fluoro-NTP, can be incorporated into the RNA molecule using T7 RNA polymerase mutants. The resistance of these modified aptamers to nuclease can be tested by incubating them with either purified nucleases or nuclease from mouse serum, and the integrity of aptamers can be analyzed by gel electrophoresis.

The monomeric or multimeric aptamer of the invention can be further attached or conjugated to a detectable or therapeutic moiety (i.e., a pharmaceutical moiety).

In one example a diagnostic moiety such as a detectable moiety e.g., label (e.g., His tag, flag tag), fluorescent, radioactive, biotin/avidin etc., can be bound to the aptamer, and imaging, immunohistochemistry, for target identification.

The present invention provides polynucleotides and salts thereof. In some examples, a polynucleotide is an oligonucleotide or a single strand of RNA or DNA. In some examples, the polynucleotide or functional fragment thereof (e.g. including a target molecule/ligand binding domain), or salt of either is an aptamer. The term “aptamer” refers to a polynucleotide or functional fragment thereof, or salt of either that specifically binds a target molecule. The term “specifically binds” is used interchangeably herein with “selectively binds” and means the same thing. As used herein the terms “specifically binds” and “selectively binds” in reference to an aptamer, describe the binding of an aptamer to a target molecule and mean that aptamer binding to the target molecule does not involve the formation of nucleotide base pairs between the aptamer and the target molecule. The skilled person would recognize it is well-known in the art that the polynucleotide sequence of an aptamer may include base pairs that are not required for specific binding of the aptamer to a given target molecule, and that smaller fragments of an aptamer, even fragments having below 50% sequence identity may still be capable of effectively binding to a target molecule (Alsager, Omar A., et al. “Ultrasensitive Colorimetric Detection of 17β-Estradiol: The Effect of Shortening DNA Aptamer Sequences.” Analytical chemistry 87.8 (2015): 4201-4209).

In some examples, an aptamer exerts an inhibitory effect on a target, e.g., by binding of the target, by catalytically altering the target, by reacting with the target in a way which modifies the target or the functional activity of the target, by ionically or covalently attaching to the target or by facilitating the reaction between the target and another molecule. The aptamer can comprise a ribonucleotide, deoxyribonucleotide, or other type of nucleic acid, or two or more different types of nucleic acids. An aptamer can also comprise one or more modified bases, sugars, polyethylene glycol spacers or phosphate backbone units. In some examples, the aptamer comprises one or more 2′ sugar modifications, such as a 2′-O-alkyl (e.g., 2′-O-methyl or 2′-O-methoxyethyl) or a 2′-fluoro modification.

In some examples, an aptamer is a polynucleotide of about 80, about 75, about 70, about 65, about 60, about 55, about 50, about 45, about 40, about 35, about 30, about 25, about 20, about 15 nucleotides in length.

In some examples, an aptamer is a polynucleotide of less than 80, less than 75, less than 70, less than 65, less than 60, less than 55, less than 50, less than 45, less than 40, less than 35, less than 30, less than 25, less than 20, less than 15 nucleotides in length.

In some examples an aptamer is a polynucleotide of about 70 to 80, is about 60 to 70, is about 50 to 60, is about 40 to 50, is about 30 to 40, is about 20 to 30, is about 10 to 20 nucleotides in length.

In some examples an aptamer is a polynucleotide of about 75 to 85, is about 65 to 75, is about 55 to 65, is about 45 to 55, is about 35 to 45, is about 25 to 35, is about 15 to 25 nucleotides in length.

In some examples an aptamer is a polynucleotide of 70 to 80, is 60 to 70, is 50 to 60, is 40 to 50, is 30 to 40, is 20 to 30, is 10 to 20 nucleotides in length.

In some examples an aptamer is a polynucleotide of 75 to 85, is 65 to 75, is 55 to 65, is 45 to 55, is 35 to 45, is 25 to 35, is 15 to 25 nucleotides in length. In some examples the aptamer is about 73 to about 77 or about 74 to about 76 nucleotides in length.

A person of skill in the art will appreciate that an aptamer may be any length polynucleotide that falls within the size parameters set out herein. By way of non-limiting example an aptamer may be about 76, about 61, about 54, about 43, about 29 or about 27 nucleotides in length or may be 77, 62, 55, 44, 28 or 26 nucleotides in length. What is important is that the aptamer specifically binds the target molecule.

In some examples an aptamer of the invention is a polynucleotide of about 80, about 75, about 70, about 65, about 60, about 55, about 50, about 45, about 40, about 35, about 30, about 25, about 20, about 15 nucleotides in length that selectively binds a target molecule as described herein.

In some examples, an aptamer is a polynucleotide of less than 80, less than 75, less than 70, less than 65, less than 60, less than 55, less than 50, less than 45, less than 40, less than 35, less than 30, less than 25, less than 20, less than 15 nucleotides in length that selectively binds a target molecule as described herein.

In some examples an aptamer is a polynucleotide of about 70 to 80, is about 60 to 70, is about 50 to 60, is about 40 to 50, is about 30 to 40, is about 20 to 30, is about 10 to 20 nucleotides in length that selectively binds a target molecule as described herein.

In some examples an aptamer is a polynucleotide of about 75 to 85, is about 65 to 75, is about 55 to 65, is about 45 to 55, is about 35 to 45, is about 25 to 35, is about 15 to 25 nucleotides in length that selectively binds a target molecule as described herein.

In some examples an aptamer is a polynucleotide of 70 to 80, is 60 to 70, is 50 to 60, is 40 to 50, is 30 to 40, is 20 to 30, is 10 to 20 nucleotides in length that selectively binds a target molecule as described herein.

In some examples an aptamer is a polynucleotide of 75 to 85, is 65 to 75, is 55 to 65, is 45 to 55, is 35 to 45, is 25 to 35, is 15 to 25 nucleotides in length that selectively binds a target molecule as described herein.

The term “salt” includes a non-toxic salt of an inorganic or organic acid, including, but not limited to, a halide (chloride, bromide, iodide, fluoride), acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, hydrochloride, hydrobromide, hydroiodide, 2-hydroxyethanesulfonate, lactate, maleate, malonate, methanesulfonate, nitrate, oxalate, persulfate, phosphate, picrate, pivalate, propionate, p-toluenesulfonate, salicylate, succinate, sulfate, tartrate, thiocyanate, and undecanoate.

The term “salt” also includes a non-toxic salt of an organic or inorganic base, including, but not limited to, sodium (Nat), potassium (Kt), calcium (Ca²⁺), magnesium (Mg²⁺), and lithium (Li⁺) salts.

In some examples, the target molecule is a small molecule. In some examples, the small molecule has a molecular weight (MW) of from about 60 to about 2000 g mol⁻¹. In other examples, the small molecule has a MW of from about 100 to about 500 g mol⁻¹. In other examples, the small molecule has a MW of from about 150 to about 350 g mol⁻¹. The molecular weight of such small molecules and the calculation of their molecular weight are well known to those of skill in the art.

### EXAMPLES

### Example 1: Aptamer Generation/Selection

The aptamers described herein were generated/selected using affinity matrix-based systematic evolution of ligands by exponential enrichment (SELEX), capture SELEX, or in silico SELEX methodologies. SELEX is a process by which a random single stranded polynucleotide library is iteratively evolved to produce sequences that can bind to a specific target.

Briefly, affinity matrix-based SELEX methodology is where a target for selection is immobilised onto a solid phase (often, sepharose beads). The immobilisation steps usually include EDC/NHS or epoxy activation chemistry, depending on the structure of the target molecule. The beads are incubated with a single-stranded random polynucleotide library at room temperature in binding and washing buffer (BWB; 20 mM Tris-HCl, pH 7.5, 100 mM NaCl, 5 mM KCl, 2 mM MgCl₂, 1 mM CaCl₂), and between 0% and 0.1% IGEPAL) for up to 18 hours. The beads are washed with 5 mL BWB, and unbound aptamers are discarded in the supernatant while target binding aptamers are retained with the beads and processed for the next round of selection. The sequences that are bound to the beads are amplified by standard amplification techniques such as polymerase chain reaction (PCR). PCR is performed using the HotMaster™ Taq kit with 200 μM dNTPs, 220 nM of forward primer, 220 nM or biotin-labelled forward primer, and 1.5 U HotMaster Taq in a final volume of 50 μL. The amplified sequences are incubated with 0.3 NaOH to separate the double stranded sequences into single stranded sequences, and the biotin-labelled strand is removed using streptavidin magnetic beads. The single stranded sequences are then incubated with the beads, as described above. This process can be repeated iteratively until the required target affinity is reached. The incubation, elution, and PCR conditions can be adjusted at each round of SELEX to ensure the sequences retain the desired binding characteristics.

Capture SELEX methodology is where target for selection is free in solution and the ssDNA library is immobilised onto beads. Briefly, a polynucleotide probe is conjugated via NHS/EDC chemistry to magnetic beads functionalised with carboxylic acid groups on the surface. A random polynucleotide library is attached to the beads by hybridization with the complementary probe. The ssDNA-beads are added to the solution containing the target and incubated at room temperature in BWB for up to 18 hours. The target-bound sequences dissociate from the probe and remain in the supernatant, while the unbound sequences remain bound to the beads which are discarded. The target-bound sequences are amplified by standard amplification techniques such as polymerase chain reaction (PCR), as described above. The amplified sequences are incubated with 0.3 NaOH to separate the double stranded sequences into single stranded sequences, and the biotin-labelled strand is removed using streptavidin magnetic beads. The resulting single stranded sequences are then hybridised to the complementary probe attached to the beads, and the next round of SELEX is carried out. This process can be repeated iteratively until the required target affinity is reached. The incubation, elution, and PCR conditions can be adjusted at each round of SELEX to ensure the sequences retain the desired binding characteristics. Affinity matrix-based and capture SELEX methodologies would be well known to those skilled in the art. For example, refer to the methodologies described in Paniel et al. (2017) Talanta 162:232-240 (capture SELEX) and Stoltenburg et al. (2012) Journal of Analytical Methods in Chemistry doi:10.1155/2012/415697 (affinity matrix SELEX), and both publications are incorporated by reference in their entirety.

The polynucleotide sequence for aptamers which selectively bind SARS-CoV-2 virus nucleocapsid protein are listed in FIGS. 1 and 2.

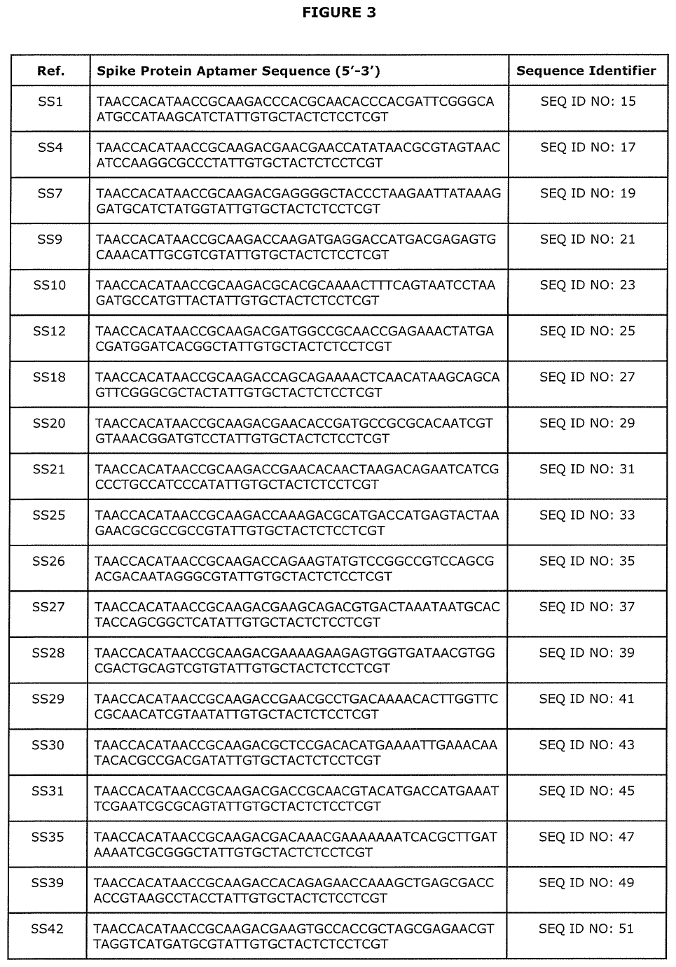

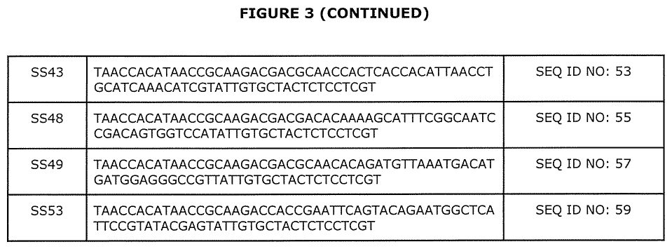

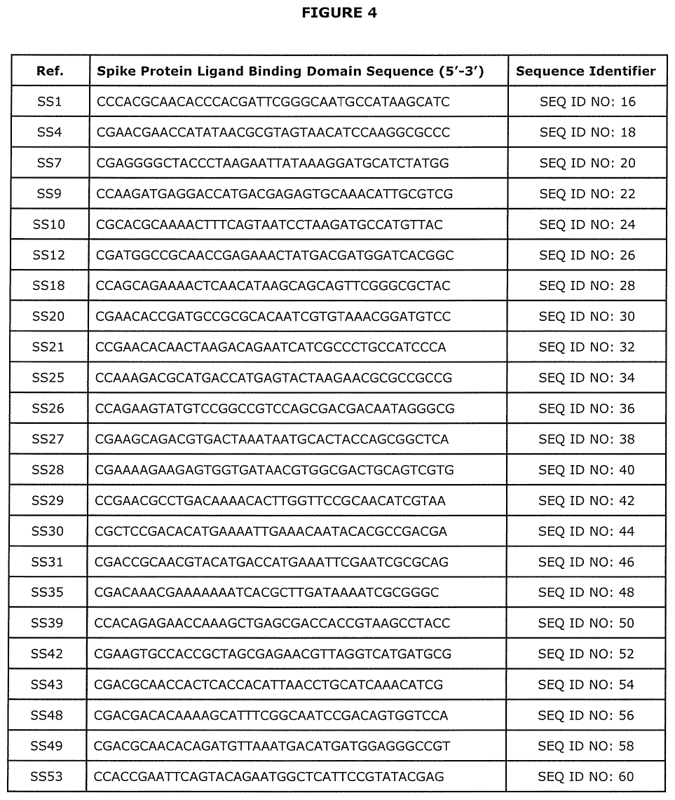

The polynucleotide sequence for aptamers which selectively bind SARS-CoV-2 virus spike protein are listed in FIGS. 3 and 4.

### Example 2: Measurement of Aptamer Binding Affinities

To determine binding affinities for the aptamers described herein, either a fluorescent imaging assay a Sybr green assay, or a fluorescent microscale thermophoresis (MST) binding assay were used.

Briefly, the fluorescent imaging assay may be used to determine Kd when the affinity matrix SELEX method is used for aptamer generation and selection. A serial dilution of hexachloro-fluorescein (HEX)-labelled aptamer is prepared with BWB. The beads with the target immobilised on the surface are washed twice with BWB before being incubated for up to 2 hours at room temperature with the fluorescently labelled aptamer candidate. Beads are then washed twice with BWB to remove loosely bound aptamers and the beads are imaged with a fluorescent microscope at 535/556 nm excitation/emission. Average fluorescence values of at least 20 beads per concentration are computed and plotted against the aptamer concentration to determine Kd.

The Sybr Green I assay may be used to determine Kd when the capture SELEX method is used for aptamer generation and selection. Sybr Green I is a fluorescent nucleic acid dye that preferentially binds to double stranded DNA. Binding of an aptamer to its target may elicit a conformational change in the aptamer structure, thereby changing the amount of double stranded regions to which the Sybr Green I dye may bind. To perform the Sybr Green I assay, a serial dilution of the target is prepared in BWB and added to a constant concentration of the unlabelled aptamer. After incubation at room temperature for up to 2 hours, a constant concentration of Sybr Green I is added and incubated for up to 2 more hours. The final concentration of aptamer and Sybr Green I are optimised to maximise the difference between a high concentration of target and no target. The fluorescence is measured at excitation/emission 480 nm/524 nm and plotted against target concentration to determine Kd.

An alternative approach to the Sybr Green I assay may be used, whereby a complementary probe is hybridised to the aptamer sequence. Addition of the target induces the aptamer to preferentially bind to the target, thereby displacing the probe and reducing the number of double stranded DNA to which the Sybr Green I dye may bind. To perform the Sybr Green I strand displacement assay, the aptamer is hybridised with the complementary probe in BWB at room temperature for up to 2 hours. A serial dilution of the target is prepared and is added to the aptamer-probe mixture and incubated at room temperature for up to 2 hours. Sybr Green I is added and incubated for up to 2 hours at room temperature. The fluorescence is measured at excitation/emission 480 nm/524 nm and plotted against target concentration to determine Kd.

The fluorescent MST binding assay may be used to determine Kd regardless of the aptamer SELEX method used. Microscale thermophoresis is based on two principles. First, the temperature related intensity changes of a fluorophore, and second, the movement of molecules along a temperature gradient. Briefly, a serial dilution of the target is prepared in BWB before being added to a Cy5-labelled aptamer. The solution is loaded into the capillaries and analysed using a Monolith NT. 115. The fluorescence was analysed using a 1:1 binding model to determine the Kd.

### Example 3: Sybr Green Assay to SARS-CoV-2 Nucleocapsid Protein

100 nM N11 (SEQ ID NO: 13) and N08 (SEQ ID NO: 7) aptamers were incubated with appropriate concentrations of the target SARS-Cov2 nucleocapsid protein for 10 mins. To the mixture, 10 μL of 100×Sybr Green Nucleic acid binding dye was added. The solution was thoroughly mixed and incubated for further 10 mins. The resulting solution was transferred into PCR tubes before the melt curve was measured using a qPCR machine. The assay is reliant on the Sybr Green dyes ability to be intercalated between the DNA forming duplex structures with any complementary nucleobases or when associating with the target molecule. The intercalated dye when excited (254 nm) has emission at 520 nm.

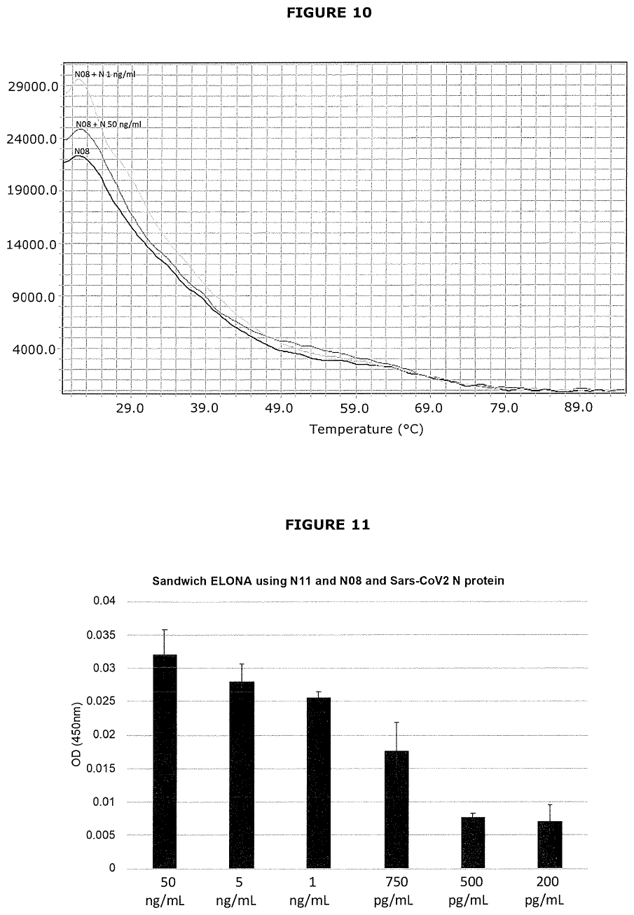

The results are presented in FIGS. 9 and 10 and reflect specific binding by N11 and N08 for the SARS-Cov2 nucleocapsid protein, with the highest resolution achieved using a sandwich assay format involving N11 (capture) and N08 (detection) aptamers at a nucleocapsid protein concentration of 50 ng/mL (FIG. 9).

### Example 4: Sandwich Elona to SARS-CoV2 Nucleocapsid Protein

Streptavidin coated 96 well please were washed thrice using carbonate bicarbonate buffer (total volume in well of 200 μL). A 100 nM 5′ biotinylated aptamer (either N11, N08, or N06 when used as primary capture) was prepared using PBS buffer. Following the plate washing step, the 100 μL of the 100 nM of the primary capture aptamer was added to each well and the plate was incubated at room temperature for 1 hr. Post incubation, the solution was discarded and washing buffer i.e. PBS was added to each well. Post adding 100 μL washing buffer, the plate was incubated with a rocking motion for 5 mins before discarding the solution. The washing step was repeated a total of three times. To measure SARS-Cov-2 nucleocaspid (N) protein, various concentrations of the N protein was prepared using the binding buffer and incubated with the primary capture aptamer for 30 mins. Following incubation with the N protein, the plate was washed three times using the binding buffer (5 mins with rocking incubation). The secondary reporting 5′ biotin labelled aptamer was prepared at 100 nM concentration in binding buffer. 100 μL of the secondary reporting aptamer (N11, N08, or N06) was added to the wells and incubated for 30 mins with a rocking motion. Following the incubation, the washing step was repeated three times before adding 50 μL of streptavidin-horse radish peroxidase (strep-HRP) to each well and incubated for 30 mins. The washing step was repeated three before adding the chemiluminescence reagent was added. Once appropriate colour was achieved, the stop solution was added, and the absorbance was read using a plate reader.

The results are presented in FIG. 11 and demonstrate dose dependent binding by the capture aptamer for SARS-Cov-2 nucleocapsid protein.

### Example 5: Lateral Flow Assay for Detection of SARs-CoV2 Nucleocapsid Protein

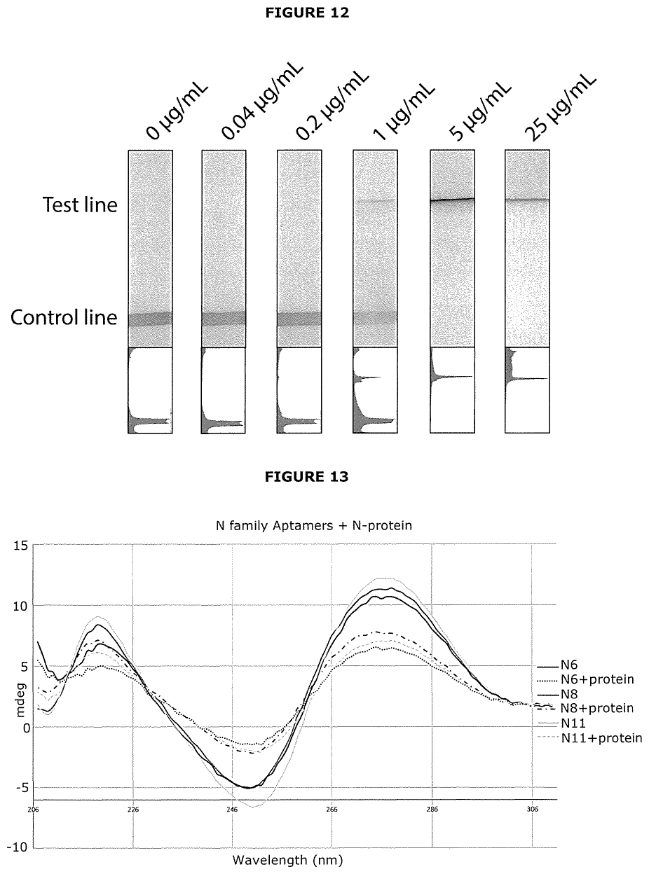

A 5′ thiol modified aptamer N11 (SEQ ID NO: 13;) was conjugated to gold nanoparticles (AuNP) using basic thiol conjugation chemistry. The AuNP-aptamer conjugate solution was added onto the sample loading pad and dried for 2 hrs at 37° C. The test line (top) and control line (bottom) are indicated on the nitrocellulose membrane. The test line constituted of 5′ biotin modified aptamer (secondary reporting aptamer) complexed with avidin protein. The control line constituted of lysozyme protein to cause the AuNP-aptamer complex to dissociate. 1 μg/mL N protein was used as a sample to test the lateral flow system. Positive result at the test line was observed when N protein was present in the spiked samples. The results are presented in FIG. 12.

### Example 6: Electrochemical Assay Using Square Wave Voltammetry for the Detection of SARs-CoV2 Nucleocapsid Protein

5′ thiol modified aptamers N06 (SEQ ID NO: 3) and N08 (SEQ ID NO: 7) were functionalised on the surface of gold electrodes using thiol conjugation chemistry. Post conjugation, the surface was blocked using mecaptahexanol (MCH) for 30 mins and washed to clean the surface. The various concentrations of the SARS-Cov-2 N protein was dispensed on the surface (5 uL) and the square wave voltemmetry (SWV) measured using a potentiostat. The change in potential for each aptamer (N06 and N08) was plotted against the concentration of the target to calculate the detection range of the assay using logarithmic (FIG. 14A) or linear (FIG. 14B) fitting. The results are presented in FIG. 14 and demonstrate that both the N06 and N08 aptamers bind the nucleocapsid protein antigen target using a structure switching “on” or structure switching “off” mechanism, with level of detection (LOD) in the pM sensitivity range.

### Example 7: Nucelocapsid Protein Detection on Universal Biosensors Proprietary Assay

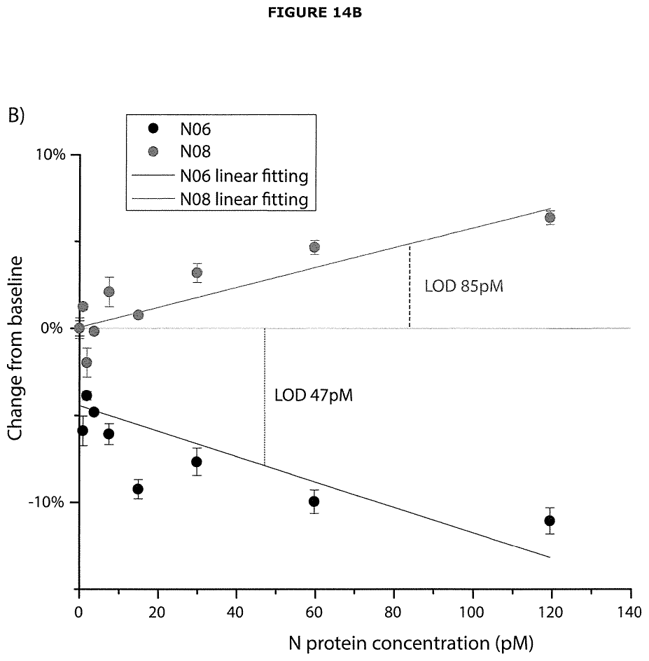

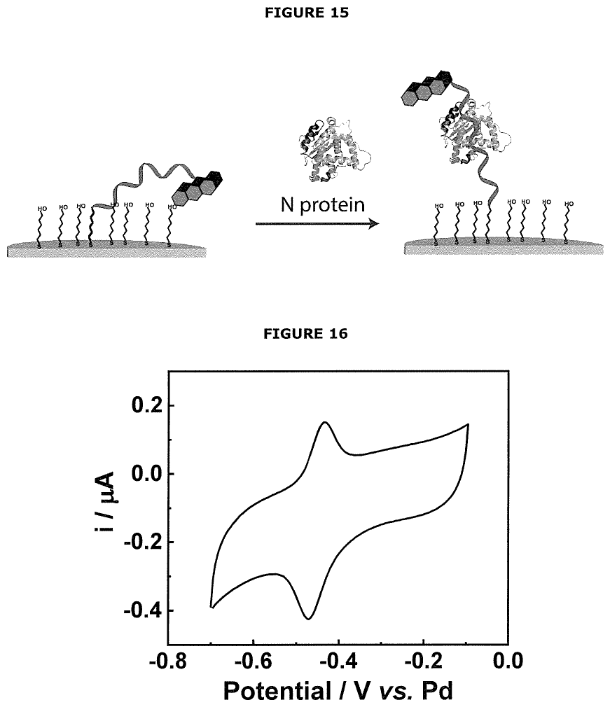

The performance of the N06 (SEQ ID NO: 3) aptamer for detection of SARS-Cov-2 nucleocapsid protein was then tested on a proprietary sensor platform developed by Universal Biosensors Pty Limited, as depicted in FIG. 15.

With reference to FIG. 15, the sensor surface contains a redox-reporter-modified nucleic acid probe that functions as a specific recognition component tethered to a gold electrode strip thiol chemistry. The electrochemical response is obtained when the target SARS-Cov-2 nucleocapsid protein binds to the N06 aptamer, reducing the efficiency with which the methylene blue redox-reporter transfers electrons to the electrode strip surface. The signal output is measured using electrochemical voltametric techniques. In this particular configuration, 6-Mercapto-1-hexanol (MCH) is used a blocking agent, that has two main purposes: (i) repel the negatively charged N06 aptamer away from the electrode surface and (ii) act as an antifouling agent.

The data presented in FIG. 16 shows a cyclic voltammogram for gold strip modified with the N06 aptamer recorded in a saliva sample from a healthy donor. As can be observed a symmetric oxidation and reduction peaks corresponding to the electrochemistry of methylene blue redox labelled at potential of approximately −460 mV with peak-to-peak separation of 40 mV is observed. This indicates that all methylene blue redox labels bonded to PNA are capable of accessing the electrodes surface within the time frame of the voltammogram at this scan rate (200 mV 5-1).

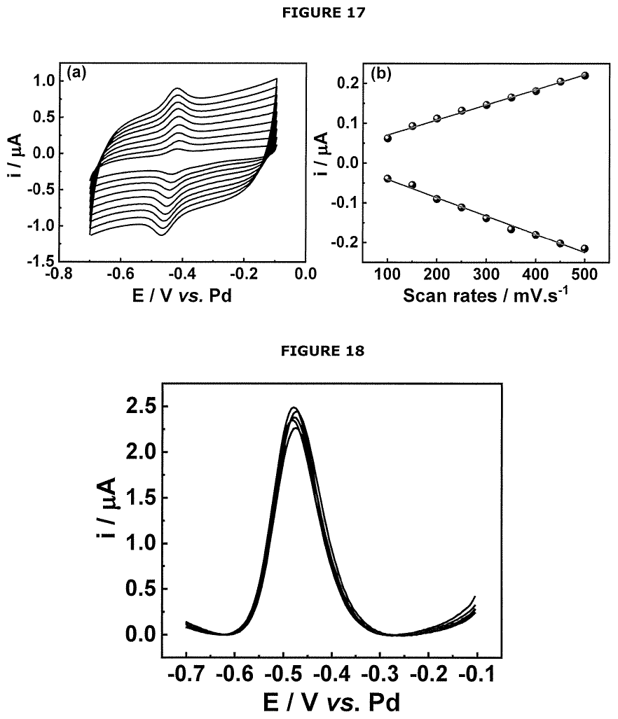

Further, the data presented in FIG. 17a show the voltammograms for the N06 aptamer recorded in saliva using different scan rates. As observed in this figure, by increasing the scan rate, there is an increase in the capacitive currents as well as faradaic currents. As seen in FIG. 17b, the anodic and cathodic currents were proportional to the scan rates in the range from 100 to 500 mV s⁻¹. This characteristic is consistent with the voltametric signal originating from a surface-confined species exhibiting ideal Nerstian behaviour.

Collectively the data presented in FIGS. 16 and 17 are important because provided is the first emperical evidence of SARS-Cov-2 nucleocapsid protein detection from a biological sample (saliva) using the NO6 (SEQ ID NO: 3) aptamer according to the present invention.

To interrogate test strip reproducibility, measurement of the N06 aptamer (SEQ ID NO: 3) was performed using five different strips prepared under the same conditions. As expected, the data presented in FIG. 18 showed very similar current magnitudes, suggesting good reproducibility in the strip preparation.

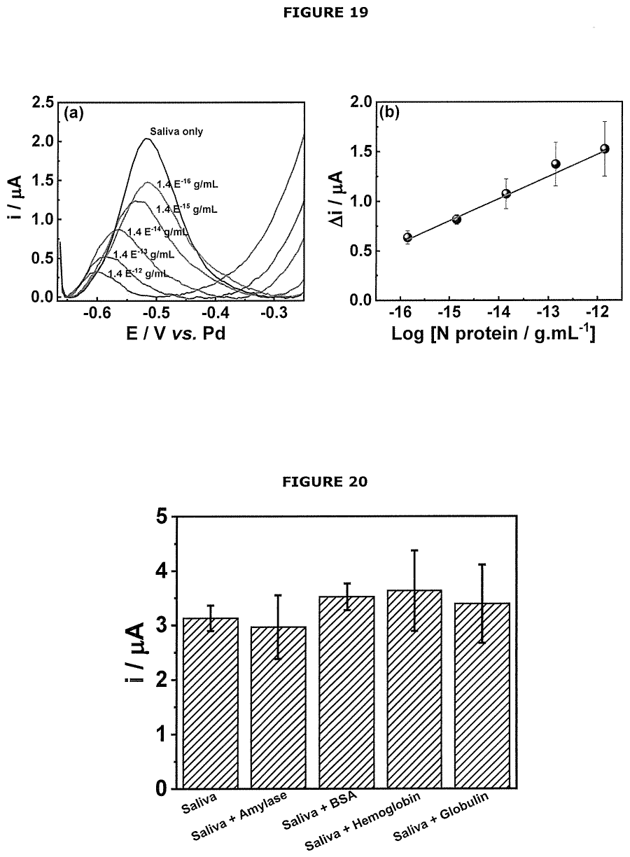

To evaluate the limit of detection and dynamic range of the N06 aptamer sensor, different concentrations of synthetic SARS-Cov-2 nucleocapsid protein were prepared using serial dilutions of protein in saliva as the medium (FIG. 19a). The saliva sample utilized in these experiments was obtained from a healthy human donor. A measurable change in the current, compared to that before the specific binding, was observed after 2 minutes exposure of the sensor to SARS-Cov-2 nucleocapsid protein solution within the concentration range of 1.4E⁻¹⁶ to 1.4E⁻¹² g/mL (FIG. 19b). The lowest detectable concentration for N protein was 1.4E⁻¹⁶ reflecting a very favourable limit of detection.

The data presented in FIG. 20 shows the electrochemical response for the N06 aptamer sensor in saliva from a healthy donor before and after spiking the sample with different biomolecules that are present in saliva and could potentially interfere with the sensor response potentially creating false positive test results. The biomolecules investigated were: amylase, bovine serum albumin (BSA), haemoglobin and globulin. According to the data presented in this figure, there was no significant variation in the electrochemical response before and after spiking the saliva sample with these biomolecules, suggesting that these molecules do not interfere with sensing performance.

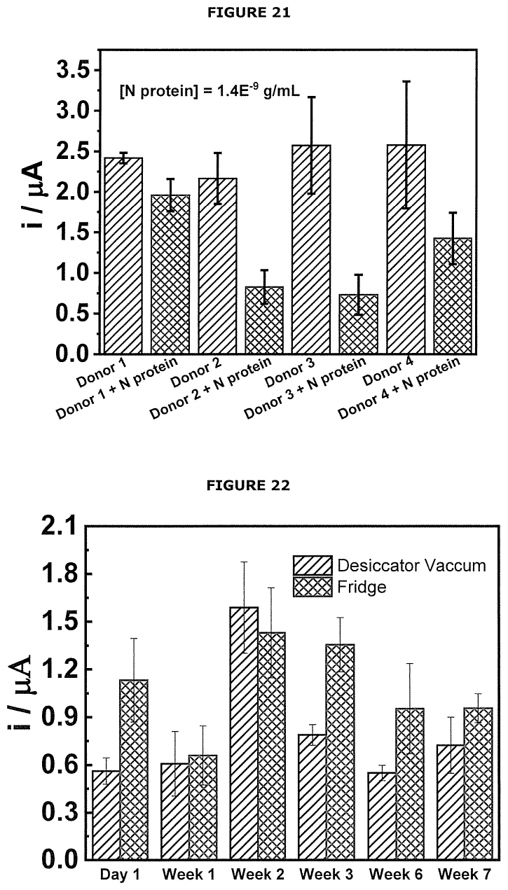

The data presented in FIG. 21 shows the electrochemical responses (current magnitude) for the measurements obtained using the N06 aptamer sensor in saliva samples from four different donors (black bars). These data reflect significant changes were obtained across the healthy donors tested. However, upon on spiking to these samples with SARS-Cov-2 nucleocapsid protein in the concentration of 1E⁻⁹ g/ml (red bars) all samples presented a reduction in electrochemical signals, reflecting that the sensor responds well to the presence of SARS-Cov-2 nucelocapsid protein.

Finally, the data presented in FIG. 22 show the extended shelf-life for the N06 aptamer sensor and that its performance was not affected by seven (7) weeks' storage in a fridge or a dessicator. These data therefore reflect that the aptamer sensor described in this example could be stored for several weeks at a time prior to use, facilitating extended shipping/transport time frames.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

1. A polynucleotide or salt thereof comprising or consisting in a sequence that has at least 75, 80, 85, 86, 87, 88, 89, 90, 91, 92, 93, 94, 95, 96, 97, 98, 99% or 100% sequence identity to any one of SEQ ID Nos: 1, 3, 5, 7, 9, 11 and 13, which polynucleotide of salt thereof selectively binds to a SARS-Cov-2 virus nucleocapsid protein.

2. The polynucleotide according to claim 1 comprising or consisting in the sequence selected from SEQ ID NO: 3, SEQ ID NO: 7 and SEQ ID NO: 13.

3. The polynucleotide according to claim 1 or claim 2 comprising or consisting in the sequence defined by SEQ ID NO: 3.
A polynucleotide or salt thereof comprising or consisting in a sequence that has at least 75, 80, 85, 86, 87, 88, 89, 90, 91, 92, 93, 94, 95, 96, 97, 98, 99% or 100% sequence identity to any one of SEQ ID Nos: 15, 17, 19, 21, 23, 25, 27, 29, 31, 33, 35, 37, 39, 41, 43, 45, 47, 49, 51, 53, 55, 57 and 59, which polynucleotide of salt thereof selectively binds to a SARS-Cov-2 virus spike protein.

4. A polynucleotide or salt thereof which binds to a SARS-CoV-2 virus nucleocapsid protein, the polynucleotide or salt thereof comprising a sequence that has at least 75, 80, 85, 86, 87, 88, 89, 90, 91, 92, 93, 94, 95, 96, 97, 98, 99 or 100% sequence identity to any one of SEQ ID Nos: 2, 4, 6, 8, 10, 12 and 14, provided that the polynucleotide has a sequence length of between about 65 and about 85 nucleotides.

5. A polynucleotide or salt thereof which binds to a SARS-CoV-2 virus spike protein, the polynucleotide comprising a sequence that has at least 75, 80, 85, 86, 87, 88, 89, 90, 91, 92, 93, 94, 95, 96, 97, 98, 99 or 100% sequence identity to any one of SEQ ID Nos: 16, 18, 20, 22, 24, 26, 28, 30, 32, 34, 36, 38, 40, 42, 44, 46, 48, 50, 52, 54, 56, 58 and 60, provided that the polynucleotide has a sequence length of between about 65 and about 85 nucleotides.

6. A polynucleotide or salt thereof comprising the sequence:
TAACCACATAACCGCAAGA[Y]TATTGTGCTACTCTCCTCGT
wherein:
(i) Y is a nucleic acid sequence comprising or consisting in any one of SEQ ID Nos: 2, 4, 6, 8, 10, 12 and 14 which polynucleotide or salt thereof selectively binds to a SARS-CoV-2 virus nucleocapsid protein; or
(ii) Y is a nucleic acid sequence comprising or consisting in any one of SEQ ID Nos: 16, 18, 20, 22, 24, 26, 28, 30, 32, 34, 36, 38, 40, 42, 44, 46, 48, 50, 52, 54, 56, 58 and 60, which polynucleotide or salt thereof selectively binds to a SARS-CoV-2 virus spike protein.

7. The polynucleotide according to any one of claims 1 to 6, wherein the binding affinity (Kd) of the polynucleotide for a SARS-Cov-2 virus antigen is between about 1 nM and about 100 uM.

8. The polynucleotide according to any one of claims 1 to 7, wherein the Gibbs Free Energy of the polynucleotide or salt thereof is between about −1.0 and about −15.0.

9. The polynucleotide or salt thereof according to any one of claims 1 to 8, wherein the polynucleotide or salt thereof is modified with a detectable label.

10. The polynucleotide according to claim 9, wherein the detectable label is a redox active moiety.

11. The polynucleotide according to claim 9 or claim 10 wherein the detectable label is methylene blue or a derivative molecule thereof.

12. A method for detecting the presence of a SARS-Cov-2 virus in a test sample, the method comprising the steps of:
(i) contacting a test sample with a polynucleotide or salt thereof comprising or consisting in a sequence defined by any one of SEQ ID Nos: 1-14, which polynucleotide selectively binds to a SARS-Cov-2 virus nucleocapsid protein; or
(ii) contacting a test sample with a polynucleotide or salt thereof comprising or consisting in a sequence defined by any one of SEQ ID Nos: 15-60, which polynucleotide selectively binds to a SARS-Cov-2 virus spike protein; and/or
(iii) contacting a test sample with a polynucleotide or salt thereof comprising or consisting in a sequence defined by any one of SEQ ID Nos: 1-14 and a polynucleotide or salt thereof comprising or consisting in a sequence defined by any one of SEQ ID Nos: 15-60; and
(iv) measuring binding between the polynucleotide and at least one SARS-Cov-2 virus antigen from the test sample,
wherein, a measured binding interaction between the polynucleotide and at least one SARS-Cov-2 virus antigen reflects the presence of a SARS-Cov-2 virus antigen in the test sample.

13. The method according to claim 12, wherein binding between the polynucleotide and at least one SARS-Cov-2 virus antigen from the test sample is performed using an aptamer modified with a redox active moiety and tethered to a metal electrode.

14. The method according to claim 13 wherein the metal electrode is a gold electrode and the presence of the SARS-Cov-2 virus antigen in the test sample is detected by measuring a change in the redox potential of the redox moiety labelled aptamer.

15. A test kit or article of manufacture for detecting the presence of a SARS-Cov-2 virus in a test sample, the test kit or article of manufacture comprising:
(i) a polynucleotide or salt thereof comprising or consisting in a sequence defined by any one of SEQ ID Nos: 1-14, which polynucleotide of salt thereof selectively binds to a SARS-Cov-2 virus nucleocapsid protein; or
(ii) a polynucleotide or salt thereof comprising a sequence defined by any one of SEQ ID Nos: 15-60, which polynucleotide of salt thereof selectively binds to a SARS-Cov-2 virus spike protein;
(iii) optionally, at least one substrate with which the aptamer is associated; and
(iv) instructions for how to detect the presence of SARS-Cov-2 virus in the test sample.


SEQUENCE LISTING (60 sequences)

SEQ ID NO: 1 - DNA, 79 bp, Artificial Sequence
  SARS-Cov-2 nucleocapsid DNA aptamer
taaccacataaccgcaagaccagtcgaagagactatttaagactccagattacccgacgtattgtgctactctcctcgt

SEQ ID NO: 2 - DNA, 40 bp, Artificial Sequence
  SARS-Cov-2 nucleocapsid DNA aptamer
ccagtcgaagagactatttaagactccagattacccgacg

SEQ ID NO: 3 - DNA, 79 bp, Artificial Sequence
  SARS-Cov-2 nucleocapsid DNA aptamer
taaccacataaccgcaagatcccgatagacccgcgtctcatgtaccttacatgctacgatattgtgctactctcctcgt

SEQ ID NO: 4 - DNA, 40 bp, Artificial Sequence
  SARS-Cov-2 nucleocapsid DNA aptamer
tcccgatagacccgcgtctcatgtaccttacatgctacga

SEQ ID NO: 5 - DNA, 79 bp, Artificial Sequence
  SARS-Cov-2 nucleocapsid DNA aptamer
taaccacataaccgcaagacgacgcgatacaaagcagtcctgaactcatgtcgcactaatattgtgctactctcctcgt

SEQ ID NO: 6 - DNA, 40 bp, Artificial Sequence
  SARS-Cov-2 nucleocapsid DNA aptamer
cgacgcgatacaaagcagtcctgaactcatgtcgcactaa

SEQ ID NO: 7 - DNA, 79 bp, Artificial Sequence
  SARS-Cov-2 nucleocapsid DNA aptamer
taaccacataaccgcaagaccacacgcacgaaaacaattgacatgaaatttacggctcttattgtgctactctcctcgt

SEQ ID NO: 8 - DNA, 40 bp, Artificial Sequence
  SARS-Cov-2 nucleocapsid DNA aptamer
ccacacgcacgaaaacaattgacatgaaatttacggctct

SEQ ID NO: 9 - DNA, 79 bp, Artificial Sequence
  SARS-Cov-2 nucleocapsid DNA aptamer
taaccacataaccgcaagacgacgcgatacaaaacactagagttctctcgcagcgcgcgtattgtgctactctcctcgt

SEQ ID NO: 10 - DNA, 40 bp, Artificial Sequence
  SARS-Cov-2 nucleocapsid DNA aptamer
cgacgcgatacaaaacactagagttctctcgcagcgcgcg

SEQ ID NO: 11 - DNA, 79 bp, Artificial Sequence
  SARS-Cov-2 nucleocapsid DNA aptamer
taaccacataaccgcaagacgaacgcaagaacgccctatatcggacacacttgggctactattgtgctactctcctcgt

SEQ ID NO: 12 - DNA, 40 bp, Artificial Sequence
  SARS-Cov-2 nucleocapsid DNA aptamer
cgaacgcaagaacgccctatatcggacacacttgggctac

SEQ ID NO: 13 - DNA, 79 bp, Artificial Sequence
  SARS-Cov-2 nucleocapsid DNA aptamer
taaccacataaccgcaagacgacgcgatacaaaacactactaatctctcgtcgcactagtattgtgctactctcctcgt

SEQ ID NO: 14 - DNA, 40 bp, Artificial Sequence
  SARS-Cov-2 nucleocapsid DNA aptamer
cgacgcgatacaaaacactactaatctctcgtcgcactag

SEQ ID NO: 15 - DNA, 79 bp, Artificial Sequence
  SARS-Cov-2 spike DNA aptamer
taaccacataaccgcaagacccacgcaacacccacgattcgggcaatgccataagcatctattgtgctactctcctcgt

SEQ ID NO: 16 - DNA, 40 bp, Artificial Sequence
  SARS-Cov-2 spike DNA aptamer
cccacgcaacacccacgattcgggcaatgccataagcatc

SEQ ID NO: 17 - DNA, 79 bp, Artificial Sequence
  SARS-Cov-2 spike DNA aptamer
taaccacataaccgcaagacgaacgaaccatataacgcgtagtaacatccaaggcgccctattgtgctactctcctcgt

SEQ ID NO: 18 - DNA, 40 bp, Artificial Sequence
  SARS-Cov-2 spike DNA aptamer
cgaacgaaccatataacgcgtagtaacatccaaggcgccc

SEQ ID NO: 19 - DNA, 79 bp, Artificial Sequence
  SARS-Cov-2 spike DNA aptamer
taaccacataaccgcaagacgaggggctaccctaagaattataaaggatgcatctatggtattgtgctactctcctcgt

SEQ ID NO: 20 - DNA, 40 bp, Artificial Sequence
  SARS-Cov-2 spike DNA aptamer
cgaggggctaccctaagaattataaaggatgcatctatgg

SEQ ID NO: 21 - DNA, 79 bp, Artificial Sequence
  SARS-Cov-2 spike DNA aptamer
taaccacataaccgcaagaccaagatgaggaccatgacgagagtgcaaacattgcgtcgtattgtgctactctcctcgt

SEQ ID NO: 22 - DNA, 40 bp, Artificial Sequence
  SARS-Cov-2 spike DNA aptamer
ccaagatgaggaccatgacgagagtgcaaacattgcgtcg

SEQ ID NO: 23 - DNA, 79 bp, Artificial Sequence
  SARS-Cov-2 spike DNA aptamer
taaccacataaccgcaagacgcacgcaaaactttcagtaatcctaagatgccatgttactattgtgctactctcctcgt

SEQ ID NO: 24 - DNA, 40 bp, Artificial Sequence
  SARS-Cov-2 spike DNA aptamer
cgcacgcaaaactttcagtaatcctaagatgccatgttac

SEQ ID NO: 25 - DNA, 79 bp, Artificial Sequence
  SARS-Cov-2 spike DNA aptamer
taaccacataaccgcaagacgatggccgcaaccgagaaactatgacgatggatcacggctattgtgctactctcctcgt

SEQ ID NO: 26 - DNA, 40 bp, Artificial Sequence
  SARS-Cov-2 spike DNA aptamer
cgatggccgcaaccgagaaactatgacgatggatcacggc

SEQ ID NO: 27 - DNA, 79 bp, Artificial Sequence
  SARS-Cov-2 spike DNA aptamer
taaccacataaccgcaagaccagcagaaaactcaacataagcagcagttcgggcgctactattgtgctactctcctcgt

SEQ ID NO: 28 - DNA, 40 bp, Artificial Sequence
  SARS-Cov-2 spike DNA aptamer
ccagcagaaaactcaacataagcagcagttcgggcgctac

SEQ ID NO: 29 - DNA, 79 bp, Artificial Sequence
  SARS-Cov-2 spike DNA aptamer
taaccacataaccgcaagacgaacaccgatgccgcgcacaatcgtgtaaacggatgtcctattgtgctactctcctcgt

SEQ ID NO: 30 - DNA, 40 bp, Artificial Sequence
  SARS-Cov-2 spike DNA aptamer
cgaacaccgatgccgcgcacaatcgtgtaaacggatgtcc

SEQ ID NO: 31 - DNA, 79 bp, Artificial Sequence
  SARS-Cov-2 spike DNA aptamer
taaccacataaccgcaagaccgaacacaactaagacagaatcatcgccctgccatcccatattgtgctactctcctcgt

SEQ ID NO: 32 - DNA, 40 bp, Artificial Sequence
  SARS-Cov-2 spike DNA aptamer
ccgaacacaactaagacagaatcatcgccctgccatccca

SEQ ID NO: 33 - DNA, 79 bp, Artificial Sequence
  SARS-Cov-2 spike DNA aptamer
taaccacataaccgcaagaccaaagacgcatgaccatgagtactaagaacgcgccgccgtattgtgctactctcctcgt

SEQ ID NO: 34 - DNA, 40 bp, Artificial Sequence
  SARS-Cov-2 spike DNA aptamer
ccaaagacgcatgaccatgagtactaagaacgcgccgccg

SEQ ID NO: 35 - DNA, 79 bp, Artificial Sequence
  SARS-Cov-2 spike DNA aptamer
taaccacataaccgcaagaccagaagtatgtccggccgtccagcgacgacaatagggcgtattgtgctactctcctcgt

SEQ ID NO: 36 - DNA, 40 bp, Artificial Sequence
  SARS-Cov-2 spike DNA aptamer
ccagaagtatgtccggccgtccagcgacgacaatagggcg

SEQ ID NO: 37 - DNA, 79 bp, Artificial Sequence
  SARS-Cov-2 spike DNA aptamer
taaccacataaccgcaagacgaagcagacgtgactaaataatgcactaccagcggctcatattgtgctactctcctcgt

SEQ ID NO: 38 - DNA, 40 bp, Artificial Sequence
  SARS-Cov-2 spike DNA aptamer
cgaagcagacgtgactaaataatgcactaccagcggctca

SEQ ID NO: 39 - DNA, 79 bp, Artificial Sequence
  SARS-Cov-2 spike DNA aptamer
taaccacataaccgcaagacgaaaagaagagtggtgataacgtggcgactgcagtcgtgtattgtgctactctcctcgt

SEQ ID NO: 40 - DNA, 40 bp, Artificial Sequence
  SARS-Cov-2 spike DNA aptamer
cgaaaagaagagtggtgataacgtggcgactgcagtcgtg

SEQ ID NO: 41 - DNA, 79 bp, Artificial Sequence
  SARS-Cov-2 spike DNA aptamer
taaccacataaccgcaagaccgaacgcctgacaaaacacttggttccgcaacatcgtaatattgtgctactctcctcgt

SEQ ID NO: 42 - DNA, 40 bp, Artificial Sequence
  SARS-Cov-2 spike DNA aptamer
ccgaacgcctgacaaaacacttggttccgcaacatcgtaa

SEQ ID NO: 43 - DNA, 79 bp, Artificial Sequence
  SARS-Cov-2 spike DNA aptamer
taaccacataaccgcaagacgctccgacacatgaaaattgaaacaatacacgccgacgatattgtgctactctcctcgt

SEQ ID NO: 44 - DNA, 40 bp, Artificial Sequence
  SARS-Cov-2 spike DNA aptamer
cgctccgacacatgaaaattgaaacaatacacgccgacga

SEQ ID NO: 45 - DNA, 79 bp, Artificial Sequence
  SARS-Cov-2 spike DNA aptamer
taaccacataaccgcaagacgaccgcaacgtacatgaccatgaaattcgaatcgcgcagtattgtgctactctcctcgt

SEQ ID NO: 46 - DNA, 40 bp, Artificial Sequence
  SARS-Cov-2 spike DNA aptamer
cgaccgcaacgtacatgaccatgaaattcgaatcgcgcag

SEQ ID NO: 47 - DNA, 78 bp, Artificial Sequence
  SARS-Cov-2 spike DNA aptamer
taaccacataaccgcaagacgacaaacgaaaaaaatcacgcttgataaaatcgcgggctattgtgctactctcctcgt

SEQ ID NO: 48 - DNA, 39 bp, Artificial Sequence
  SARS-Cov-2 spike DNA aptamer
cgacaaacgaaaaaaatcacgcttgataaaatcgcgggc

SEQ ID NO: 49 - DNA, 79 bp, Artificial Sequence
  SARS-Cov-2 spike DNA aptamer
taaccacataaccgcaagaccacagagaaccaaagctgagcgaccaccgtaagcctacctattgtgctactctcctcgt

SEQ ID NO: 50 - DNA, 40 bp, Artificial Sequence
  SARS-Cov-2 spike DNA aptamer
ccacagagaaccaaagctgagcgaccaccgtaagcctacc

SEQ ID NO: 51 - DNA, 79 bp, Artificial Sequence
  SARS-Cov-2 spike DNA aptamer
taaccacataaccgcaagacgaagtgccaccgctagcgagaacgttaggtcatgatgcgtattgtgctactctcctcgt

SEQ ID NO: 52 - DNA, 40 bp, Artificial Sequence
  SARS-Cov-2 spike DNA aptamer
cgaagtgccaccgctagcgagaacgttaggtcatgatgcg

SEQ ID NO: 53 - DNA, 79 bp, Artificial Sequence
  SARS-Cov-2 spike DNA aptamer
taaccacataaccgcaagacgacgcaaccactcaccacattaacctgcatcaaacatcgtattgtgctactctcctcgt

SEQ ID NO: 54 - DNA, 40 bp, Artificial Sequence
  SARS-Cov-2 spike DNA aptamer
cgacgcaaccactcaccacattaacctgcatcaaacatcg

SEQ ID NO: 55 - DNA, 79 bp, Artificial Sequence
  SARS-Cov-2 spike DNA aptamer
taaccacataaccgcaagacgacgacacaaaagcatttcggcaatccgacagtggtccatattgtgctactctcctcgt

SEQ ID NO: 56 - DNA, 40 bp, Artificial Sequence
  SARS-Cov-2 spike DNA aptamer
cgacgacacaaaagcatttcggcaatccgacagtggtcca

SEQ ID NO: 57 - DNA, 79 bp, Artificial Sequence
  SARS-Cov-2 spike DNA aptamer
taaccacataaccgcaagacgacgcaacacagatgttaaatgacatgatggagggccgttattgtgctactctcctcgt

SEQ ID NO: 58 - DNA, 40 bp, Artificial Sequence
  SARS-Cov-2 spike DNA aptamer
cgacgcaacacagatgttaaatgacatgatggagggccgt

SEQ ID NO: 59 - DNA, 79 bp, Artificial Sequence
  SARS-Cov-2 spike DNA aptamer
taaccacataaccgcaagaccaccgaattcagtacagaatggctcattccgtatacgagtattgtgctactctcctcgt

SEQ ID NO: 60 - DNA, 40 bp, Artificial Sequence
  SARS-Cov-2 spike DNA aptamer
ccaccgaattcagtacagaatggctcattccgtatacgag